(19) United States
(12) Patent Application Publication
(10) Pub. No.: US 2012/0023602 A1
(43) Pub. Date: Jan. 26, 2012

(54) INTERMARKET CLASS HYBRID LETTUCE

(75) Inventors: George D. Gibson, Prunedale, CA (US); Nathan K. Olivas, Carmel Valley, CA (US); Peter Salm, Capitola, CA (US)

(73) Assignee: PROGENY ADVANCED GENETICS, Salinas, CA (US)

(21) Appl. No.: 12/808,361

(22) PCT Filed: Dec. 19, 2008
(86) PCT No.: PCT/US08/87789

§ 371 Date: Aug. 2, 2011

Related U.S. Application Data
(60) Provisional application No. 61/015,108, filed on Dec. 19, 2007.

(51) Int. Cl. A01H 5/00; C12N 5/04; A01H 1/02; A01H 5/10

(52) U.S. Cl. 800/260; 800/305; 435/410

(57) ABSTRACT

Methods for producing intermarket class hybrid lettuce seed are described. The methods include the steps of providing lettuce plants and releasing Megachile rotundata bees or attracting pollinators. Intermarket class hybrid lettuce seeds and plants produced using the disclosed methods, and male sterile lettuce lines used in the methods, are also described.

### RELATED APPLICATION

This application claims the benefit of U.S. Provisional Application No. 61/015,108, filed Dec. 19, 2007, which is hereby incorporated by reference.

### BACKGROUND

Lactuca sativa, L., commonly known as lettuce, is an increasingly popular crop. Worldwide lettuce consumption continues to increase. Part of the increase in consumption can be attributed to the expanding processes and salad mixes that are now available. No longer is lettuce only available as a whole head Iceberg, Romaine, Green Leaf and Butter, but new unique types of lettuce are available as are an array of packaged salad blends and mixes that add to its growing popularity and convenience. As a result of this demand, and as a result of the evolving ways in which lettuce is grown, harvested and processed, there is a continued need for new innovative lettuce varieties, new innovative lettuce types, and new innovative lettuce morphologies.

### BRIEF SUMMARY

New intermarket class lettuce F1 hybrids are described herein. The present disclosure thus relates to the seeds of intermarket class hybrids, to the plants of hybrid lettuce and to methods for producing intermarket hybrids from the hybridization or crossing of two different market classes of lettuce. The lettuce plants are members of the species Lactuca sativa or Lactuca serriola. The market classes described herein include but are not limited to Iceberg, Romaine, Green Leaf, Red Leaf, Batavia and Butter lettuce plants. The intermarket class hybrids described herein are uniform F1 hybrids. They exhibit qualities and characteristics of their parents and are consistently larger than one or both parents.

An embodiment provides for an intermarket class hybrid lettuce plant seed derived from a cross between a female lettuce plant of a first market class and a male lettuce plant of a second market class where the seed produces an intermarket class hybrid lettuce plant having an average increase in fresh head weight at maturity of at least 15%, at least 20%, at least 25%, at least 30%, at least 35%, at least 40%, at least 45%, at least 50%, at least 55%, at least 60%, at least 65%, or at least 70% as compared to the average fresh head weight of the female lettuce plant of the first market class at maturity or as compared to the average fresh head weight of the male lettuce plant of the second market class at maturity. The intermarket class hybrid lettuce plant seed produces uniform plants. In one embodiment, the female and the male lettuce plant are inbred plants.

An additional embodiment provides for an intermarket class hybrid lettuce plant or part thereof produced by growing the seed of any of the preceding crosses. It further provides for a lettuce plant having all of the physiological and morphological characteristics of the intermarket class hybrid lettuce plant. A further embodiment may include the pollen of the intermarket class hybrid lettuce plant. Another embodiment may include a tissue culture of the intermarket class hybrid lettuce plant.

An embodiment provides for an intermarket class hybrid lettuce plant seed derived from a cross between a female Iceberg lettuce plant and a male Romaine lettuce plant where the seed produces an intermarket class hybrid lettuce plant having an average increase in fresh head weight at maturity of at least 15%, at least 20%, at least 25%, at least 30%, at least 35%, at least 40%, or at least 45% as compared to the average fresh head weight of the female Iceberg lettuce plant at maturity.

A further embodiment provides for an intermarket class hybrid lettuce plant seed derived from a cross between a female Iceberg lettuce plant and a male Romaine lettuce plant where the seed produces an intermarket class hybrid lettuce plant having an average increase in fresh head weight at maturity of at least 15%, at least 20%, at least 25%, at least 30%, at least 35%, at least 40% or at least 45%, at least 50%, at least 55%, at least 60%, at least 65%, at least 70%, or at least 72% as compared to the average fresh head weight of the male Romaine lettuce plant at maturity.

An additional embodiment provides for an intermarket class hybrid lettuce plant or part thereof produced by growing the seed of a female Iceberg and male Romaine cross. It further provides for a lettuce plant having all of the physiological and morphological characteristics of the intermarket class hybrid lettuce plant produced from a female Iceberg and male Romaine cross. A further embodiment may include the pollen of the intermarket class hybrid lettuce plant derived from the female Iceberg and male Romaine cross. Yet a further embodiment may include a tissue culture of the intermarket class hybrid lettuce plant derived from the female Iceberg and male Romaine cross.

An embodiment provides for an intermarket class hybrid lettuce plant seed derived from a cross between a female Romaine lettuce plant and a male Iceberg lettuce plant where the seed produces an intermarket class hybrid lettuce plant having an average increase in fresh head weight at maturity of at least 15%, at least 20%, at least 25%, at least 30%, at least 35%, at least 40%, at least 45%, or at least 50% as compared to the average fresh head weight of the female Romaine lettuce plant at maturity.

A further embodiment provides for an intermarket class hybrid lettuce plant seed derived from a cross between a female Romaine lettuce plant and a male Iceberg lettuce plant where the seed produces an intermarket class hybrid lettuce plant having an average increase in fresh head weight at maturity of at least 15%, at least 20%, at least 25%, at least 30%, at least 35%, at least 40%, at least 45%, or at least 47% as compared to the average fresh head weight of the male Iceberg lettuce plant at maturity.

An additional embodiment provides for an intermarket class hybrid lettuce plant or part thereof produced by growing the seed of a female Romaine and male Iceberg cross. It further provides for a lettuce plant having all of the physiological and morphological characteristics of the intermarket class hybrid lettuce plant produced from a female Romaine and male Iceberg cross. A further embodiment may include the pollen of the intermarket class hybrid lettuce plant derived from the female Romaine and male Iceberg cross. Yet a further embodiment may include a tissue culture of the intermarket class hybrid lettuce plant derived from the female Romaine and male Iceberg cross.

An embodiment provides for an intermarket class hybrid lettuce plant seed derived from a cross between a female Romaine lettuce plant and a male Green Leaf lettuce plant where the seed produces an intermarket class hybrid lettuce plant having an average increase in fresh head weight at maturity of at least 15% or at least 18% as compared to the average fresh head weight of the female Romaine lettuce plant at maturity.

A further embodiment provides for an intermarket class hybrid lettuce plant seed derived from a cross between a female Romaine lettuce plant and a male Green Leaf lettuce plant where the seed produces an intermarket class hybrid lettuce plant having an average increase in fresh head weight at maturity of at least 15%, at least 20%, at least 25%, at least 30%, at least 35%, at least 40%, at least 45%, at least 50%, or at least 54% as compared to the average fresh head weight of the male Green Leaf lettuce plant at maturity.

An additional embodiment provides for an intermarket class hybrid lettuce plant or part thereof produced by growing the seed of a female Romaine and male Green Leaf cross. It further provides for a lettuce plant having all of the physiological and morphological characteristics of the intermarket class hybrid lettuce plant produced from a female Romaine and male Green Leaf cross. A further embodiment may include the pollen of the intermarket class hybrid lettuce plant derived from the female Romaine and male Green Leaf cross. Yet a further embodiment may include a tissue culture of the intermarket class hybrid lettuce plant derived from the female Romaine and male Green Leaf cross.

An embodiment provides for an intermarket class hybrid lettuce plant seed derived from a cross between a female Green Leaf lettuce plant and a male Romaine lettuce plant where the seed produces an intermarket class hybrid lettuce plant having an average increase in fresh head weight at maturity of at least 15%, at least 20%, or at least 25% as compared to the average fresh head weight of the female Green Leaf lettuce plant at maturity.

An additional embodiment provides for an intermarket class hybrid lettuce plant or part thereof produced by growing the seed of a female Green Leaf and male Romaine cross. It further provides for a lettuce plant having all of the physiological and morphological characteristics of the intermarket class hybrid lettuce plant produced from a female Green Leaf and male Romaine cross. A further embodiment may include the pollen of the intermarket class hybrid lettuce plant derived from the female Green Leaf and male Romaine cross. Yet a further embodiment may include a tissue culture of the intermarket class hybrid lettuce plant derived from the female Green Leaf and male Romaine cross.

A further embodiment provides for an intermarket class hybrid lettuce plant seed derived from a cross between a female Iceberg lettuce plant and a male Green Leaf lettuce plant where the seed produces an intermarket class hybrid lettuce plant having an average increase in fresh head weight at maturity of at least 15%, at least 20%, at least 25%, at least 30%, at least 35%, at least 40%, at least 45%, at least 50%, at least 55%, or at least 58% as compared to the average fresh head weight of the male Green Leaf lettuce plant at maturity.

An additional embodiment provides for an intermarket class hybrid lettuce plant or part thereof produced by growing the seed of a female Iceberg and male Green Leaf cross. It further provides for a lettuce plant having all of the physiological and morphological characteristics of the intermarket class hybrid lettuce plant produced from a female Iceberg and male Green Leaf cross. A further embodiment may include the pollen of the intermarket class hybrid lettuce plant derived from the female Iceberg and male Green Leaf cross. Yet a further embodiment may include a tissue culture of the intermarket class hybrid lettuce plant derived from the female Iceberg and male Green Leaf cross.

An embodiment provides for an intermarket class hybrid lettuce plant seed derived from a cross between a female Green Leaf lettuce plant and a male Iceberg lettuce plant where the seed produces an intermarket class hybrid lettuce plant having an average increase in fresh head weight at maturity of at least 15%, at least 20%, or at least 25%, at least 30%, or at least 35% as compared to the average fresh head weight of the female Green Leaf lettuce plant at maturity.

A further embodiment provides for an intermarket class hybrid lettuce plant seed derived from a cross between a female Green Leaf lettuce plant and a male Iceberg lettuce plant where the seed produces an intermarket class hybrid lettuce plant having an average increase in fresh head weight at maturity of at least 15%, at least 20%, at least 25%, at least 30%, at least 35%, at least 40%, at least 45%, at least 50%, at least 55%, at least 60%, at least 65%, at least 70%, or at least 74% as compared to the average fresh head weight of the male Iceberg lettuce plant at maturity.

An additional embodiment provides for an intermarket class hybrid lettuce plant or part thereof produced by growing the seed of a female Green Leaf and male Iceberg cross. It further provides for a lettuce plant having all of the physiological and morphological characteristics of the intermarket class hybrid lettuce plant produced from a female Green Leaf and male Iceberg cross. A further embodiment may include the pollen of the intermarket class hybrid lettuce plant derived from the female Green Leaf and male Iceberg cross. Yet a further embodiment may include a tissue culture of the intermarket class hybrid lettuce plant derived from the female Green Leaf and male Iceberg cross.

An embodiment provides for an intermarket class hybrid lettuce plant seed derived from a cross between a female Iceberg lettuce plant and a male Butter lettuce plant where the seed produces an intermarket class hybrid lettuce plant having an average increase in fresh head weight at maturity of at least 12% as compared to the average fresh head weight of the female Iceberg lettuce plant at maturity.

A further embodiment provides for an intermarket class hybrid lettuce plant seed derived from a cross between a female Iceberg lettuce plant and a male Butter lettuce plant where the seed produces an intermarket class hybrid lettuce plant having an average increase in fresh head weight at maturity of at least 15%, at least 20%, at least 25%, at least 30%, at least 35%, at least 40%, at least 45%, at least 50%, at least 55%, at least 60%, or at least 61% as compared to the average fresh head weight of the male Butter lettuce plant at maturity.

An additional embodiment provides for an intermarket class hybrid lettuce plant or part thereof produced by growing the seed of a female Iceberg and male Butter cross. It further provides for a lettuce plant having all of the physiological and morphological characteristics of the intermarket class hybrid lettuce plant produced from a female Iceberg and male Butter cross. A further embodiment may include the pollen of the intermarket class hybrid lettuce plant derived from the female Iceberg and male Butter cross. Yet a further embodiment may include a tissue culture of the intermarket class hybrid lettuce plant derived from the female Iceberg and male Butter cross.

An embodiment provides for an intermarket class hybrid lettuce plant seed derived from a cross between a female Romaine lettuce plant and a male Butter lettuce plant where the seed produces an intermarket class hybrid lettuce plant having an average increase in fresh head weight at maturity of at least 15%, at least 20%, at least 25%, at least 30%, at least 35%, or at least 36% as compared to the average fresh head weight of the female Romaine lettuce plant at maturity.

A further embodiment provides for an intermarket class hybrid lettuce plant seed derived from a cross between a female Romaine lettuce plant and a male Butter lettuce plant where the seed produces an intermarket class hybrid lettuce plant having an average increase in fresh head weight at maturity of at least 15%, at least 20%, at least 25%, at least 30%, at least 35%, or at least 37% as compared to the average fresh head weight of the male Butter lettuce plant at maturity.

An additional embodiment provides for an intermarket class hybrid lettuce plant or part thereof produced by growing the seed of a female Romaine and male Butter cross. It further provides for a lettuce plant having all of the physiological and morphological characteristics of the intermarket class hybrid lettuce plant produced from a female Romaine and male Butter cross. A further embodiment may include the pollen of the intermarket class hybrid lettuce plant derived from the female Romaine and male Butter cross. Yet a further embodiment may include a tissue culture of the intermarket class hybrid lettuce plant derived from the female Romaine and male Butter cross.

A method is provided for producing intermarket class hybrid lettuce seed, including the steps of: a) providing a first inbred lettuce plant from the group of lettuce market classes including Iceberg, Romaine, Green Leaf, Red Leaf, Batavia and Butter lettuce plants, where the first inbred lettuce plant provides pollen; b) providing a second inbred lettuce plant from the group of lettuce market classes including Iceberg, Romaine, Green Leaf, Red Leaf, Batavia and Butter lettuce plants where the second inbred lettuce plant is emasculated; c) transferring the pollen from the first inbred lettuce plant to the second inbred lettuce plant to produce a pollinated inbred lettuce plant where the first inbred lettuce plant is from a different market class than the market class of the second inbred lettuce plant; and d) growing the pollinated lettuce plant to produce intermarket class hybrid lettuce seed.

A further embodiment may include providing the first lettuce plant by planting seed of the first lettuce plant where the seed germinates and grows into the inbred lettuce plant. In yet a further embodiment, the emasculated lettuce plant may be a male sterile lettuce plant.

An embodiment is provided where the first lettuce plant is an Iceberg lettuce plant and the second lettuce plant is a Romaine lettuce plant.

An embodiment is provided where the first lettuce plant is an Iceberg lettuce plant and the second lettuce plant is a Green Leaf lettuce plant.

An embodiment is provided where the first lettuce plant is an Iceberg lettuce plant and the second lettuce plant is a Red Leaf lettuce plant.

An embodiment is provided where the first lettuce plant is an Iceberg lettuce plant and the second lettuce plant is a Batavia lettuce plant.

An embodiment is provided where the first lettuce plant is an Iceberg lettuce plant and the second lettuce plant is a Butter lettuce plant.

An embodiment is provided where the first lettuce plant is a Romaine lettuce plant and the second lettuce plant is an Iceberg lettuce plant.

An embodiment is provided where the first lettuce plant is a Romaine lettuce plant and the second lettuce plant is a Green Leaf lettuce plant.

An embodiment is provided where the first lettuce plant is a Romaine lettuce plant and the second lettuce plant is a Red Leaf lettuce plant.

An embodiment is provided where the first lettuce plant is a Romaine lettuce plant and the second lettuce plant is a Batavia lettuce plant.

An embodiment is provided where the first lettuce plant is a Romaine lettuce plant and the second lettuce plant is a Butter lettuce plant.

An embodiment is provided where the first lettuce plant is a Butter lettuce plant and the second lettuce plant is an Iceberg lettuce plant.

An embodiment is provided where the first lettuce plant is a Butter lettuce plant and the second lettuce plant is a Romaine lettuce plant.

An embodiment is provided where the first lettuce plant is a Butter lettuce plant and the second lettuce plant is a Green Leaf lettuce plant.

An embodiment is provided where the first lettuce plant is a Butter lettuce plant and the second lettuce plant is a Red Leaf lettuce plant.

An embodiment is provided where the first lettuce plant is a Butter lettuce plant and the second lettuce plant is a Batavia lettuce plant.

An embodiment is provided where the first lettuce plant is a Red Leaf lettuce plant and the second lettuce plant is an Iceberg lettuce plant.

An embodiment is provided where the first lettuce plant is a Red Leaf lettuce plant and the second lettuce plant is a Romaine lettuce plant.

An embodiment is provided where the first lettuce plant is a Red Leaf lettuce plant and the second lettuce plant is a Green Leaf lettuce plant.

An embodiment is provided where the first lettuce plant is a Red Leaf lettuce plant and the second lettuce plant is a Batavia lettuce plant.

An embodiment is provided where the first lettuce plant is a Red Leaf lettuce plant and the second lettuce plant is a Butter lettuce plant.

An embodiment is provided where the first lettuce plant is a Green Leaf lettuce plant and the second lettuce plant is an Iceberg lettuce plant.

An embodiment is provided where the first lettuce plant is a Green Leaf lettuce plant and the second lettuce plant is a Romaine lettuce plant.

An embodiment is provided where the first lettuce plant is a Green Leaf lettuce plant and the second lettuce plant is a Red Leaf lettuce plant.

An embodiment is provided where the first lettuce plant is a Green Leaf lettuce plant and the second lettuce plant is a Batavia lettuce plant.

An embodiment is provided where the first lettuce plant is a Green Leaf lettuce plant and the second lettuce plant is a Butter lettuce plant.

An embodiment is provided where the first lettuce plant is a Batavia lettuce plant and the second lettuce plant is an Iceberg lettuce plant.

An embodiment is provided where the first lettuce plant is a Batavia lettuce plant and the second lettuce plant is a Romaine lettuce plant.

An embodiment is provided where the first lettuce plant is a Batavia lettuce plant and the second lettuce plant is a Green Leaf lettuce plant.

An embodiment is provided where the first lettuce plant is a Batavia lettuce plant and the second lettuce plant is a Red Leaf lettuce plant.

An embodiment is provided where the first lettuce plant is a Batavia lettuce plant and the second lettuce plant is a Butter lettuce plant.

An embodiment is further provided where step c) is performed by Megachile bees.

A method for producing intermarket class hybrid lettuce seed is provided, comprising the steps of: a) providing a first inbred lettuce plant from the group of lettuce market classes including Iceberg, Romaine, Green Leaf, Red Leaf, Batavia and Butter lettuce plants where the first inbred lettuce plant provides pollen; b) providing a second inbred lettuce plant selected from the group of lettuce market classes including Iceberg, Romaine, Green Leaf, Red Leaf, Batavia and Butter lettuce plants where the second inbred lettuce plant is emasculated; c) releasing Megachile bees, where the bees transfer pollen from the first inbred lettuce plant to the second inbred lettuce to produce a pollinated inbred lettuce plant where the first inbred lettuce plant is from a different market class than the market class of the second inbred lettuce plant; and d) growing the pollinated lettuce plant to produce hybrid lettuce seed.

The Megachile bee may be Megachile rotundata, Megachile relative, or Megachile pugnata. In a preferred embodiment the Megachile bee is Megachile rotundata.

A further embodiment may include providing the first lettuce plant by planting seed of the first lettuce plant where the seed germinates and grows into the inbred lettuce plant. The emasculated lettuce plant may be a lettuce plant that does not produce pollen and providing of the emasculated lettuce plant may be by planting seed of the emasculated lettuce plant where the seed germinates and grows into the emasculated lettuce plant.

An embodiment is provided where the releasing of the bees may be performed in an enclosed area or an open field.

A further embodiment is provided where the first lettuce plant and the emasculated plant flower during a flowering period where the releasing step is performed by releasing a batch of bees on a weekly basis during the flowering period.

The step of releasing may be performed at a temperature of at least about 74° F.

An embodiment is provided where the first lettuce plant is an Iceberg lettuce plant and the second lettuce plant is a Romaine lettuce plant.

An embodiment is provided where the first lettuce plant is an Iceberg lettuce plant and the second lettuce plant is a Green Leaf lettuce plant.

An embodiment is provided where the first lettuce plant is an Iceberg lettuce plant and the second lettuce plant is a Red Leaf lettuce plant.

An embodiment is provided where the first lettuce plant is an Iceberg lettuce plant and the second lettuce plant is a Batavia lettuce plant.

An embodiment is provided where the first lettuce plant is an Iceberg lettuce plant and the second lettuce plant is a Butter lettuce plant.

An embodiment is provided where the first lettuce plant is a Romaine lettuce plant and the second lettuce plant is an Iceberg lettuce plant.

An embodiment is provided where the first lettuce plant is a Romaine lettuce plant and the second lettuce plant is a Green Leaf lettuce plant.

An embodiment is provided where the first lettuce plant is a Romaine lettuce plant and the second lettuce plant is a Red Leaf lettuce plant.

An embodiment is provided where the first lettuce plant is a Romaine lettuce plant and the second lettuce plant is a Batavia lettuce plant.

An embodiment is provided where the first lettuce plant is a Romaine lettuce plant and the second lettuce plant is a Butter lettuce plant.

An embodiment is provided where the first lettuce plant is a Butter lettuce plant and the second lettuce plant is an Iceberg lettuce plant.

An embodiment is provided where the first lettuce plant is a Butter lettuce plant and the second lettuce plant is a Romaine lettuce plant.

An embodiment is provided where the first lettuce plant is a Butter lettuce plant and the second lettuce plant is a Green Leaf lettuce plant.

An embodiment is provided where the first lettuce plant is a Butter lettuce plant and the second lettuce plant is a Red Leaf lettuce plant.

An embodiment is provided where the first lettuce plant is a Butter lettuce plant and the second lettuce plant is a Batavia lettuce plant.

An embodiment is provided where the first lettuce plant is a Red Leaf lettuce plant and the second lettuce plant is an Iceberg lettuce plant.

An embodiment is provided where the first lettuce plant is a Red Leaf lettuce plant and the second lettuce plant is a Romaine lettuce plant.

An embodiment is provided where the first lettuce plant is a Red Leaf lettuce plant and the second lettuce plant is a Green Leaf lettuce plant.

An embodiment is provided where the first lettuce plant is a Red Leaf lettuce plant and the second lettuce plant is a Batavia lettuce plant.

An embodiment is provided where the first lettuce plant is a Red Leaf lettuce plant and the second lettuce plant is a Butter lettuce plant.

An embodiment is provided where the first lettuce plant is a Green Leaf lettuce plant and the second lettuce plant is an Iceberg lettuce plant.

An embodiment is provided where the first lettuce plant is a Green Leaf lettuce plant and the second lettuce plant is a Romaine lettuce plant.

An embodiment is provided where the first lettuce plant is a Green Leaf lettuce plant and the second lettuce plant is a Red Leaf lettuce plant.

An embodiment is provided where the first lettuce plant is a Green Leaf lettuce plant and the second lettuce plant is a Batavia lettuce plant.

An embodiment is provided where the first lettuce plant is a Green Leaf lettuce plant and the second lettuce plant is a Butter lettuce plant.

An embodiment is provided where the first lettuce plant is a Batavia lettuce plant and the second lettuce plant is an Iceberg lettuce plant.

An embodiment is provided where the first lettuce plant is a Batavia lettuce plant and the second lettuce plant is a Romaine lettuce plant.

An embodiment is provided where the first lettuce plant is a Batavia lettuce plant and the second lettuce plant is a Green Leaf lettuce plant.

An embodiment is provided where the first lettuce plant is a Batavia lettuce plant and the second lettuce plant is a Red Leaf lettuce plant.

An embodiment is provided where the first lettuce plant is a Batavia lettuce plant and the second lettuce plant is a Butter lettuce plant.

An embodiment provides for an intermarket class hybrid lettuce seed resulting from a cross between a first lettuce plant providing pollen and an emasculated lettuce plant, where the cross is performed by the steps of: a) transferring pollen from the first lettuce plant to the emasculated lettuce plant to form a pollinated lettuce plant where the first and second lettuce plants are from the group of lettuce market classes including of Iceberg, Romaine, Green Leaf, Red Leaf, Batavia and Butter lettuce plants where the first and second lettuce plants are from different market classes; and b) growing the pollinated lettuce plant to produce intermarket class hybrid lettuce seed.

A further embodiment is provided where the seed produces a plant having an average head weight greater than the average head weight of the first lettuce plant.

An embodiment is provided where the first lettuce plant is an Iceberg lettuce plant and the second lettuce plant is a Romaine lettuce plant.

An embodiment is provided where the first lettuce plant is an Iceberg lettuce plant and the second lettuce plant is a Green Leaf lettuce plant.

An embodiment is provided where the first lettuce plant is an Iceberg lettuce plant and the second lettuce plant is a Red Leaf lettuce plant.

An embodiment is provided where the first lettuce plant is an Iceberg lettuce plant and the second lettuce plant is a Batavia lettuce plant.

An embodiment is provided where the first lettuce plant is an Iceberg lettuce plant and the second lettuce plant is a Butter lettuce plant.

An embodiment is provided where the first lettuce plant is a Romaine lettuce plant and the second lettuce plant is an Iceberg lettuce plant.

An embodiment is provided where the first lettuce plant is a Romaine lettuce plant and the second lettuce plant is a Green Leaf lettuce plant.

An embodiment is provided where the first lettuce plant is a Romaine lettuce plant and the second lettuce plant is a Red Leaf lettuce plant.

An embodiment is provided where the first lettuce plant is a Romaine lettuce plant and the second lettuce plant is a Batavia lettuce plant.

An embodiment is provided where the first lettuce plant is a Romaine lettuce plant and the second lettuce plant is a Butter lettuce plant.

An embodiment is provided where the first lettuce plant is a Butter lettuce plant and the second lettuce plant is an Iceberg lettuce plant.

An embodiment is provided where the first lettuce plant is a Butter lettuce plant and the second lettuce plant is a Romaine lettuce plant.

An embodiment is provided where the first lettuce plant is a Butter lettuce plant and the second lettuce plant is a Green Leaf lettuce plant.

An embodiment is provided where the first lettuce plant is a Butter lettuce plant and the second lettuce plant is a Red Leaf lettuce plant.

An embodiment is provided where the first lettuce plant is a Butter lettuce plant and the second lettuce plant is a Batavia lettuce plant.

An embodiment is provided where the first lettuce plant is a Red Leaf lettuce plant and the second lettuce plant is an Iceberg lettuce plant.

An embodiment is provided where the first lettuce plant is a Red Leaf lettuce plant and the second lettuce plant is a Romaine lettuce plant.

An embodiment is provided where the first lettuce plant is a Red Leaf lettuce plant and the second lettuce plant is a Green Leaf lettuce plant.

An embodiment is provided where the first lettuce plant is a Red Leaf lettuce plant and the second lettuce plant is a Batavia lettuce plant.

An embodiment is provided where the first lettuce plant is a Red Leaf lettuce plant and the second lettuce plant is a Butter lettuce plant.

An embodiment is provided where the first lettuce plant is a Green Leaf lettuce plant and the second lettuce plant is an Iceberg lettuce plant.

An embodiment is provided where the first lettuce plant is a Green Leaf lettuce plant and the second lettuce plant is a Romaine lettuce plant.

An embodiment is provided where the first lettuce plant is a Green Leaf lettuce plant and the second lettuce plant is a Red Leaf lettuce plant.

An embodiment is provided where the first lettuce plant is a Green Leaf lettuce plant and the second lettuce plant is a Batavia lettuce plant.

An embodiment is provided where the first lettuce plant is a Green Leaf lettuce plant and the second lettuce plant is a Butter lettuce plant.

An embodiment is provided where the first lettuce plant is a Batavia lettuce plant and the second lettuce plant is an Iceberg lettuce plant.

An embodiment is provided where the first lettuce plant is a Batavia lettuce plant and the second lettuce plant is a Romaine lettuce plant.

An embodiment is provided where the first lettuce plant is a Batavia lettuce plant and the second lettuce plant is a Green Leaf lettuce plant.

An embodiment is provided where the first lettuce plant is a Batavia lettuce plant and the second lettuce plant is a Red Leaf lettuce plant.

An embodiment is provided where the first lettuce plant is a Batavia lettuce plant and the second lettuce plant is a Butter lettuce plant.

An embodiment provides for an intermarket class hybrid lettuce seed resulting from a cross between a first lettuce plant providing pollen and an emasculated lettuce plant, where the cross is performed by a process comprising the steps of: a) releasing Megachile bees, where the bees transfer pollen from the first lettuce plant to the emasculated lettuce plant to form a pollinated lettuce plant where the first and second lettuce plants are selected from a group of lettuce market classes including Iceberg, Romaine, Green Leaf, Red Leaf, Batavia and Butter lettuce plants where the first and second lettuce plants are from different market classes; and b) growing the pollinated lettuce plant to produce intermarket class hybrid lettuce seed.

A further embodiment is provided where the seed produces a plant having an average head weight greater than the average head weight of the first lettuce plant.

An embodiment is provided where the first lettuce plant is an Iceberg lettuce plant and the second lettuce plant is a Romaine lettuce plant.

An embodiment is provided where the first lettuce plant is an Iceberg lettuce plant and the second lettuce plant is a Green Leaf lettuce plant.

An embodiment is provided where the first lettuce plant is an Iceberg lettuce plant and the second lettuce plant is a Red Leaf lettuce plant.

An embodiment is provided where the first lettuce plant is an Iceberg lettuce plant and the second lettuce plant is a Batavia lettuce plant.

An embodiment is provided where the first lettuce plant is an Iceberg lettuce plant and the second lettuce plant is a Butter lettuce plant.

An embodiment is provided where the first lettuce plant is a Romaine lettuce plant and the second lettuce plant is an Iceberg lettuce plant.

An embodiment is provided where the first lettuce plant is a Romaine lettuce plant and the second lettuce plant is a Green Leaf lettuce plant.

An embodiment is provided where the first lettuce plant is a Romaine lettuce plant and the second lettuce plant is a Red Leaf lettuce plant.

An embodiment is provided where the first lettuce plant is a Romaine lettuce plant and the second lettuce plant is a Batavia lettuce plant.

An embodiment is provided where the first lettuce plant is a Romaine lettuce plant and the second lettuce plant is a Butter lettuce plant.

An embodiment is provided where the first lettuce plant is a Butter lettuce plant and the second lettuce plant is an Iceberg lettuce plant.

An embodiment is provided where the first lettuce plant is a Butter lettuce plant and the second lettuce plant is a Romaine lettuce plant.

An embodiment is provided where the first lettuce plant is a Butter lettuce plant and the second lettuce plant is a Green Leaf lettuce plant.

An embodiment is provided where the first lettuce plant is a Butter lettuce plant and the second lettuce plant is a Red Leaf lettuce plant.

An embodiment is provided where the first lettuce plant is a Butter lettuce plant and the second lettuce plant is a Batavia lettuce plant.

An embodiment is provided where the first lettuce plant is a Red Leaf lettuce plant and the second lettuce plant is an Iceberg lettuce plant.

An embodiment is provided where the first lettuce plant is a Red Leaf lettuce plant and the second lettuce plant is a Romaine lettuce plant.

An embodiment is provided where the first lettuce plant is a Red Leaf lettuce plant and the second lettuce plant is a Green Leaf lettuce plant.

An embodiment is provided where the first lettuce plant is a Red Leaf lettuce plant and the second lettuce plant is a Batavia lettuce plant.

An embodiment is provided where the first lettuce plant is a Red Leaf lettuce plant and the second lettuce plant is a Butter lettuce plant.

An embodiment is provided where the first lettuce plant is a Green Leaf lettuce plant and the second lettuce plant is an Iceberg lettuce plant.

An embodiment is provided where the first lettuce plant is a Green Leaf lettuce plant and the second lettuce plant is a Romaine lettuce plant.

An embodiment is provided where the first lettuce plant is a Green Leaf lettuce plant and the second lettuce plant is a Red Leaf lettuce plant.

An embodiment is provided where the first lettuce plant is a Green Leaf lettuce plant and the second lettuce plant is a Batavia lettuce plant.

An embodiment is provided where the first lettuce plant is a Green Leaf lettuce plant and the second lettuce plant is a Butter lettuce plant.

An embodiment is provided where the first lettuce plant is a Batavia lettuce plant and the second lettuce plant is an Iceberg lettuce plant.

An embodiment is provided where the first lettuce plant is a Batavia lettuce plant and the second lettuce plant is a Romaine lettuce plant.

An embodiment is provided where the first lettuce plant is a Batavia lettuce plant and the second lettuce plant is a Green Leaf lettuce plant.

An embodiment is provided where the first lettuce plant is a Batavia lettuce plant and the second lettuce plant is a Red Leaf lettuce plant.

An embodiment is provided where the first lettuce plant is a Batavia lettuce plant and the second lettuce plant is a Butter lettuce plant.

A further embodiment is provided where the intermarket class lettuce plants may be Lactuca sativa or Lactuca serriola species.

### DETAILED DESCRIPTION OF PREFERRED EMBODIMENTS

Described herein is a system for the effective production of commercial quantities of hybrid lettuce seed. A series of uniform intermarket class lettuce hybrids are also described.

In one aspect, an embodiment provides lettuce plants including a lettuce plant of a specified market class with pollen and an emasculated lettuce plant from a different market class, thus enabling the production of an intermarket class hybrid plant. The lettuce plant with pollen can either be an inbred line or an F1 line and can be any plant from the species of Lactuca with characteristics that are desired in a hybrid plant or with a genotype that is expected to produce a hybrid plant with desired characteristics. In one embodiment, provided is a method for producing intermarket class F1 lettuce hybrid seeds by transferring pollen from a plant of a specific market class to an emasculated lettuce plant of a different market class.

In another aspect, an embodiment provides methods for pollinating an emasculated lettuce plant of a specified market class by providing a first lettuce plant of a different market class having pollen; providing at least one emasculated lettuce plant of a specified market class; and releasing bees, where the bees transfer pollen from the first lettuce plant to the emasculated lettuce plant to pollinate the emasculated lettuce plant. In a preferred embodiment, the bees are Megachile bees. The emasculated plant may either be a plant that does not produce pollen or a plant where the pollen has been removed. In preferred embodiments, the emasculated plant is a plant that does not produce pollen, most preferably, a male sterile line. In other embodiments, the emasculated plant is a plant where the pollen has been removed, e.g., by another removal or misting.

In a further aspect, an embodiment provides methods for pollinating an emasculated lettuce plant of a specified market class as described above with the additional step of attracting Megachile bees.

These Megachile bees may be attracted by positioning one or more attractors in the proximity of the lettuce plants, where the Megachile bees transfer pollen from the first lettuce plant to the emasculated lettuce plant. Alternatively, the Megachile bees may be attracted by positioning plants or plant products which attract the Megachile bees in the proximity of the lettuce plants. The attractor is preferably alfalfa.

In yet another aspect, an embodiment provides methods for producing intermarket class hybrid lettuce seed. In one preferred embodiment, the method has the steps of providing a first lettuce plant of a specified market class having pollen; providing at least one emasculated lettuce plant from a different market class; and releasing Megachile bees, where the bees transfer pollen from the first lettuce plant to the emasculated lettuce plant to form a pollinated lettuce plant; and growing the pollinated lettuce plant to produce intermarket class hybrid lettuce seed. In another preferred embodiment, the method has the steps of providing a first lettuce plant of a specified market class having pollen; providing at least one emasculated lettuce plant from a different market class; and attracting Megachile bees to the first lettuce plant, where the Megachile bees transfer pollen from the first lettuce plant to the emasculated lettuce plant to form a pollinated lettuce plant; and growing the pollinated lettuce plant to produce intermarket class hybrid lettuce seed.

In still yet another aspect, an embodiment provides methods for pollinating an emasculated lettuce plant having the steps of: providing a first lettuce plant of a specified market class having pollen; providing at least one emasculated lettuce plant from a different market class; and attracting Megachile bees to the first lettuce plant, where the Megachile bees transfer pollen from the first lettuce plant to the emasculated lettuce plant and form a pollinated lettuce.

In preferred embodiments of methods, the Megachile bee is Megachile rotundata, Megachile relativa, or Megachile pugnata. The first lettuce plant can be an inbred or F1 line of a specified market class. In preferred embodiments, the emasculated plant is a male sterile breeding line (also known as female line) from a different market class. The lettuce plants are preferably Iceberg, Romaine, Red Leaf, Butter, Serriola or Green Leaf cultivars.

The release of the bees can be performed in an open field or an enclosed area. The bees may be released in batches on a weekly basis, e.g., 50,000 or 100,000/batch. In preferred embodiments, the step of releasing is performed in a climate that reaches at least about 74° F. during the time that the lettuce flower bloom.

In other aspects, an embodiment provides intermarket class hybrid lettuce seed. In one preferred embodiment, the hybrid lettuce seed results from a cross between a first lettuce plant of a specified market class having pollen and an emasculated lettuce plant from a different market class, where the cross (pollination) is performed by a process having the steps of releasing Megachile bees, where the bees transfer pollen from the first lettuce plant to the emasculated lettuce plant to form a pollinated lettuce plant; and growing the pollinated lettuce plant to produce intermarket class hybrid lettuce seed. In another preferred embodiment, the intermarket class hybrid lettuce seed results from a cross between a first lettuce plant of a specified market class having pollen and an emasculated plant from a different market class, where the cross is performed by a process having the steps of attracting Megachile bees, where the Megachile bees transfer pollen from the first lettuce plant to the emasculated lettuce plant to form a pollinated lettuce plant; and growing the pollinated lettuce plant to produce intermarket class hybrid lettuce seed.

Emasculated lettuce plants can be provided by transplanting emasculated lettuce plants, transplanting lettuce plants and emasculating them or by planting seeds which germinate and grow into emasculated lettuce plants or plants that can be emasculated. Plants having pollen can be provided by transplanting lettuce plants with pollen, transplanting lettuce plants which grow to produce pollen or by planting seeds which germinate into emasculated lettuce plants.

The present disclosure is further directed to a method for producing intermarket class hybrid lettuce seed, including the steps of: a) providing a first inbred lettuce plant chosen from Iceberg, Romaine, Green Leaf, Red Leaf, Batavia and Butter market classes where the first inbred lettuce plant includes pollen; b) providing a second inbred lettuce plant chosen from Iceberg, Romaine, Green Leaf, Red Leaf, Batavia and Butter market classes where the second inbred lettuce plant is emasculated; c) transferring the pollen from the first inbred lettuce plant to the second inbred lettuce plant to produce a pollinated inbred lettuce plant where the first inbred lettuce plant is from a different market class than the market class of the second inbred lettuce plant; and d) growing the pollinated lettuce plant to produce intermarket class hybrid lettuce seed.

The present disclosure is further directed to intermarket class hybrids based on crosses between Iceberg, Romaine, Green Leaf, Red Leaf, Batavia and Butter market classes as describe in more detail below.

### DEFINITIONS

As used herein, the term “lettuce” refers to any cultivated member of the Lactuca genus including Lactuca sativa L. and Lactuca serriola species. Lettuce does not refer to wild lettuce, Lactuca Canadensis.

As used herein, the term “releasing” refers to any act requiring human intervention which results in movement of bees from a space lacking cultivated lettuce plants to one with cultivated lettuce plants present.

As used herein, the term “positioning” refers to the placement of plants and or plant-derived products at a distance from lettuce plants that are short enough to attract bees and increase the frequency of pollination of lettuce plants.

As used herein, the term “proximity” refers to being within a sufficiently small distance from lettuce to attract bees and increase the frequency of pollination of lettuce plants.

As used herein, the term “attracting” refers to any act which is conducted with the purpose of or the effect of increasing the number and/or concentration of lettuce pollinators near lettuce plants.

The term “emasculated” refers to cultivated lettuce plants where pollen is removed or not produced. Lettuce may be emasculated via methods known in the art, including classical breeding to develop male sterile plants, engineered male-sterility, anther tube removal, and pollen washing. Such emasculated plants are also known as male sterile plants or female plants. It is understood that cultivated male sterile lettuce plants may produce a small amount of pollen. Such plants may produce a small amount of seed via self pollination.

The term “market class” lettuce refers to the type and growth habit of the lettuce. For instance, a market class can be, but is not limited to, Iceberg, Romaine, Green Leaf, Red Leaf, Butter, or Batavia type lettuces. An intermarket class F1 hybrid is an F1 hybrid product that intercrosses two distinct types of market class lettuce to make a product that expresses the traits of both market classes.

### Methods for Pollination and Hybrid Lettuce Seed Production

Methods for pollinating emasculated cultivated lettuce plants and producing hybrid lettuce seeds are provided herein.

### Lettuce

The lettuce plants can be any cultivated member of the Lactuca market species capable of being pollinated by methods described herein. Preferred lettuce types include Iceberg, Romaine, Green Leaf, Red Leaf, Butter Batavia and Serriola.

In preferred embodiments of the methods described herein seeds of cultivated lettuce plants capable of producing pollen and lettuce plants incapable of producing pollen or plants from which pollen will be removed prior to pollination (emasculated or to be emasculated lettuce plants) are planted in the vicinity of one another to enable the production of intermarket class hybrid lettuce plants through use of the pollination procedures described herein. The lettuce plant capable of producing pollen and used to donate pollen can either be an inbred line or an F1 line and can be any species of Lactuca sativa or Lactuca serriola with characteristics that are desired in a hybrid plant or with a genotype that is expected to produce a hybrid plant with desired characteristics.

The emasculated lettuce plants are lettuce plants where pollen is removed or not produced. Pollen can be removed by removing the anthers or misting the anthers to wash off pollen. This process of misting is a proven and effective means of pollen removal that assures crossing or hybridization. About 60-90 minutes past sunrise, flowers to be used for crossings are selected. The basis for selection are open flowers, with the stigma emerged and the pollen visibly attached to the single stigma (about 10-20 stigma). Using 3-4 pumps of water from a regular spray bottle, the pollen is washed off with enough pressure to dislodge the pollen grains, but not enough to damage the style. Excess water is dried off with clean paper towels. About 30 minutes later, the styles should spring back up and the two lobes of the stigma will be visibly open in a “V” shape. Anthers can be removed using any method known to those of skill in the art. One method is to pinch the side of the anther cone of an unopened flower with tweezers and pull the anther straight out.

The emasculated lettuce plant can include lettuce emasculated via methods known in the art, including, but not limited to, male-sterility, anther tube removal, and pollen washing. In preferred embodiments, the emasculated plant is any of the male sterile lettuce lines known in the art, such as those described by Ryder. The male sterility may be cytoplasmic, genetic, cytoplasmic-genetic, functional, or due to self-incompatibility. In a particularly preferred embodiment, the male sterile line used is one of a distinct market class from that of the pollinating parent.

Lettuce lines which do not produce pollen (or produce insignificant amounts of pollen) can also be utilized to produce intermarket class hybrid lettuce seeds and plants. Such emasculated plants can be a male sterile lettuce plant of a specified market class. The male sterility may be cytoplasmic, genetic, cytoplasmic-genetic, functional, or result from self-incompatibility. Such emasculated plants are also known as female lettuce plants or male sterile plants. Plants that produce pollen are also known as male plants. Male sterile lettuce lines are available. For example, the male sterile line MS7×Salinas 88, BC4 is available from Dr. Edward Ryder at the USDA Salinas Calif. research facility. MS7 was first described in Ryder, Genetic Studies in Lettuce (Lactuca sativa L.), J. Amer. Soc. Hort. Sci. (1971) 96(6): 826-828.

MS7×Salinas 88, BC4 can be utilized to produce additional male sterile lines via crossing with cultivated lettuce varieties. For example, Iceberg, Romaine, Red Leaf, Serriola or Green Leaf cultivated lettuce types can be crossed with MS7×Salinas 88, BC4 to produce F1 seed. In one embodiment, F1 seed is planted and cuttings are taken and maintained in tissue culture. Sterile plants are identified at the flowering stage. Sterile plants are increased in tissue culture. The tissue cultured plants are transferred to soil where the plants are grown to the flowering stage and then crossed with pollen-producing flowers to produce seed. Seed is harvested. The harvested seed is planted and the resulting plants are further selected and rogued for uniformity and the desired traits. Additional back crossing can be performed to increase the homozygosity of the male sterile mother lines. This process can be repeated. Once such male sterile lines are stable and uniform, they can be used as male sterile plants to produce hybrid lettuce seed by crossing with pollen-producing inbred lettuce plants using the methods described herein.

### Pollination

Pollination can be performed by any means, such as by hand using techniques well known in the art. Alternatively, pollination can be facilitated by the use of certain bees. The methods described herein include the step of releasing bees of the Megachile species. Preferably the bees are Megachile rotundata, Megachile pugnata, or Megachile relative bees. While not wishing to be bound by theory, it is believed that bees of the Megachile species are particularly useful for methods described herein because aspects of its behavior allow it to effectively pollinate lettuce. For example, under preferred temperatures, the Megachile is active during the short morning period in which lettuce flowers are open and capable of being pollinated. Furthermore, the Megachile is a small and agile bee that does not appear to be hindered by the heavy sticky latex present on the stems and branches of the flowering lettuce plant. This small size also allows the bee to forage deep inside the small lettuce flowers, thus permitting pollination to occur. Megachile have been observed foraging on the lettuce flowers and show no preference between male sterile or male fertile flowers. In addition, Megachile return to their nest each evening, so that once released, they do not just fly off to another location. Furthermore, they do not communicate with each other as honey bees do, so they are less likely to leave in mass numbers in search of other more lucrative pollen/nectar sources.

Megachile bees are solitary bees meaning that each female lays eggs and provisions her own nest cells. Even though they are solitary, the Megachile bee is also a gregarious bee which means it prefers to live close to other bees of the same species.

Megachile bees will generally pollinate at temperatures of approximately 74 to 80° F. or above as they prefer dry sunny climates; these bees will not pollinate as well in cool cloudy or rainy weather. Rearing of Megachile bees is well established; the pupae are relatively low cost to purchase.

Megachile bees are available from International Pollination Systems Inc. who obtains such bees from commercial suppliers in Canada and the western U.S. such as Idaho. Megachile bees are generally delivered as late instar larvae enclosed in leaf cells. Cells are generally sold in gallon quantities with one gallon containing about 10,000 cells. High quality cells will generally result in about 80% bee emergence. The Megachile bee cells are generally stored in screen trays or vented pint glass jars at 40° F. (4° C.). Cells should be kept in layers of approximately 3.8 cm or less to minimize reduced bee emergence; greater cell depths allow overheating which kills larvae in the bottom layer of cells. The bee cells require about 30 day warm treatment before all bees will emerge as described at the USDA Web site on pollination. Such warm up periods are generally carried out in an incubator.

The release of bees is timed to optimize pollen transfer from one lettuce plant to another by the bees. Preferably, the bees are released at the time when the lettuce plants are mature and in the initial flowering stage. Since it takes a period of time for bees to become acclimated to the environment and become effective pollinators, the bees should be released slightly before the beginning of the optimal time period for pollination.

The bees are released in the vicinity of the provided lettuce plants. The bees may be released any distance from the lettuce plants with pollen for which the bees will travel to reach the lettuce and perform the transfer of pollen from one lettuce plant to another. This distance may be from about zero to about 200 feet from the bee hive to the plant. In certain embodiments, the distance is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, and about 200 feet. Preferably, bee nesting boards are placed in a southward orientation to face the sun throughout the day.

The bees can be released in any type of environment which supports the growth of the lettuce plants and the movement of the bees for a time sufficient to transfer pollen. Typical environments for release include an open field or an enclosed space, such as a screened cage or a greenhouse. The temperature of the environment is one that reaches at least approximately 74° F. during the time that the lettuce flowers bloom.

In all environments, the conditions should be such that the bees are likely to transfer pollen from one lettuce plant to another rather than between other plants of other species. Preferably, the site of bee release is located away from other plants attractive to the bees and/or the site of release is a location with a low proportion of other plants attractive to the bees.

The number and frequency of bees released should be such that the density of bees is high enough to ensure sufficient pollination to achieve fertilization. Preferably, the density is high enough to generate sufficient F1 lettuce seed. Preferably, the density of bees is approximately 100,000 bees/acre. If the density of bees drops below the desired level, then additional bees can be released.

The preferred frequency of release will vary depending on the particular environment. For example, in screen cage and field conditions, the bees are preferably released in batches on a weekly basis. The number of bees released will also vary depending on the particular environment. For example, when bees are released in an open field of roughly 2 acres, a population of at least approximately 200,000 Megachile bees should be maintained throughout the flowering cycle.

### Planting of Seeds of Cultivated Lettuce Plants

In preferred embodiments, seeds of cultivated lettuce plants utilized to produce intermarket class hybrid lettuce seed are planted at a sufficient distance away from other flowering crops (or at a different time) to minimize the possibility of Megachile bees favoring flower species over lettuce. If the Megachile bees were to favor flower species other than lettuce, the pollination rate for the lettuce plants could be decreased.

In preferred embodiments, the seeds of the cultivated lettuce plants are planted so that there is a higher proportion of female (emasculated or male sterile) of a specified market class to male (pollen-producing) plants from a different market class. Lettuce plants are generally grown in rows. In certain embodiments, the ratio of male to female rows is 1 male:1 female in alternating rows or 1 male:2 female in rows.

Inter-Class Hybrid Lettuce Seed

Male sterile lines may be utilized in the development and breeding of hybrid lettuce varieties. The male sterility (lack of pollen) of the female parent minimizes competition between self and foreign pollen, thus increasing the rate of production of intermarket class F1 hybrid seed. The intermarket F1 seed is produced as the pollen from the male parent (pollen producing plant) of a specified market class is transferred, for example, via Megachile bee, to the pollen-free stigmas of the female (male sterile) parent from a different market class.

In one embodiment, a field is planted with alternate rows of seeds of male fertile and male sterile lettuce plants. The lettuce plants are grown, allowed to bolt, and flower. At the first stages of flowering the male sterile rows are rogued and the segregating male fertile plants are removed from the rows containing male sterile lines. The male fertile plants are identified by the presence of pollen inside the composite flower. In one format, the Megachile bee nesting boxes are placed on posts, elevated from the ground the edge of the field, with the nest tube openings facing south. In one format, a nesting box is placed approximately every 100 feet along the north side of the field. The first release of Megachile bees is generally designed to obtain a population of bees equivalent to 100,000 Megachile bees per acre. Additional Megachile bees can be released to maintain this population throughout the flowering period of the male sterile plants. Once flowering significantly subsides in the male sterile plants, the male fertile plants are removed from the field to prevent seed contamination. Once the seed is set and the plants have dried sufficiently, the intermarket class F1 seed produced is harvested from the male sterile lettuce flowers. The intermarket class F1 seed is cleaned and processed and planted to produce intermarket class F1 hybrid lettuce plants.

The use of a male sterile line eliminates the need to emasculate one of the parents in order to reduce selfing. For example, instead of washing pollen from stigma and manually transferring pollen to the de-pollinated maternal parent, hybrid seed production can be achieved in a single step by transferring pollen to a male sterile parent either manually or by use of Megachile bees. In one embodiment, hybrid lettuce plants are produced by providing an inbred male sterile line as described herein and another inbred pollen producing line and releasing Megachile bees to transfer pollen from the inbred pollen producing male parent to the male sterile female line.

In one embodiment, intermarket class hybrid lettuce seeds and plants are produced from use of the herein described methods. A lettuce plant of a specified market class having pollen and an emasculated plant from a different market class are provided and Megachile bees are released. Pollen is transferred from the lettuce plant with pollen to the emasculated plant by the bee. Fertilization produces intermarket class F1 hybrid seeds. Intermarket class hybrid lettuce plants are grown from these intermarket class F1 seeds.

Intermarket F1 hybrid lettuce plants are those lettuce plants produced from the first filial generation after flowers from two genetically different or two distinct market class parent lines are cross pollinated. Such intermarket class hybrid lettuce plants are said to result from a cross. In preferred embodiments, the intermarket class hybrid lettuce plants are those with improved growth parameters as compared to the parent plant, or hybrid vigor. Hybrid vigor is also referred to as heterosis. Most preferably, the hybrid lettuce plants are those with an increase in average fresh head weight at maturity over the parent lines. The increase in average fresh head weight at maturity can be at least about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, or about 162%. Such percentages are measured on a fresh head weight basis. Measurements are made at market maturity where each plant is cut and trimmed to market specifications and then weighed to the nearest gram. In other embodiments, the plants are those with improved per acre head yield or increased fresh weight in terms of pounds or tons total.

Attraction of Megachile Bees in Order to Produce Intermarket Class Hybrid Plants

Methods of pollinating lettuce and producing intermarket class hybrid lettuce which include the step of attracting bees are also provided. The step of attracting bees may be the only step in the method or may be one of several steps in the methods of pollinating cultivated lettuce. For example, a method for pollinating lettuce can include releasing Megachile bees, attracting Megachile bees or a combination thereof.

Bees, which may include Megachile bees, can be attracted to lettuce by any method used by those of skill in the art to attract bees to a particular location. For example, plants which are known to be attractive to Megachile bees, such as alfalfa can be grown near the lettuce. Alternatively, products derived from the plant containing the components of the plant which attract the Megachile bees can be placed near or in contact with the lettuce plant. When the lettuce plants flower, the alfalfa plants can be disked down or the plant products removed so that the Megachile bees will focus on the lettuce plants. Such products can also be sprayed on the lettuce plants. Wild Megachile bees can also be attracted by creating an environment that is attractive to them, such as nesting boards or tubes for nesting.

In one embodiment, a field is planted with alternate rows of seeds of male fertile and male sterile lettuce plants and hedging rows of alfalfa in order to attract Megachile bees. The lettuce plants are grown, allowed to bolt, and flower. At the first stages of flowering, the male sterile rows are rogued and the segregating male fertile plants are removed from the rows containing male sterile lines. The male fertile plants are identified by the presence of pollen inside the composite flower. Once flowering significantly subsides in the male sterile plants, the male fertile plants are removed from the field to prevent seed contamination. Once the seed is set and the plants have dried sufficiently, the intermarket class F1 seed produced is harvested from the male sterile lettuce flowers. The intermarket class F1 seed is cleaned and processed and planted to produce intermarket F1 hybrid lettuce plants.

The description will be better understood by reference to the following Examples. All of the references cited herein are hereby incorporated by reference.

### EXAMPLES

### Example 1

### Origin and Breeding History of Male Sterile Lines

Dr. Edward Ryder has described several male sterility systems, but each has characteristics which hinder its use commercially.

Ryder described an epistatically controlled pollen sterile male in “An Epistatically Controlled Pollen Sterile in Lettuce,” Ryder, Edward. American Society for Horticultural Science. Vol. 83, 1963. In this system, more than one gene is involved in controlling sterility. Therefore, there are less predictable segregation ratios, making it difficult to predict the number of male sterile plants available to produce seed. In addition, the sterile plants remain partially fertile, making it difficult to guarantee a high rate of hybridization in the F1 generation. There is also no obvious differentiation between the sterile and fertile plants until the flowering stage.

A recessive male sterility gene was first described in “Recessive Male Sterility Gene in Lettuce,” Ryder, Edward. American Society for Horticultural Science. Vol. 91, 1967. In this system, male sterile plants are partially female sterile, decreasing the number of seeds set per flower. The male sterile must undergo a self-pollination generation following each back cross-generation in order to regain male sterility. This doubles the development time to back cross the sterility gene into adapted female parent varieties. Since this is a recessive sterility system you only get 25% male sterile plants, making it necessary to rogue 75% of the plants from a seed production crop.

A male dominant male sterile line was described in “Genetic Studies in Lettuce: Male Sterility, as controlled by a single dominant gene Ms7 ms7,” Ryder, Edward. American Society for Horticultural Science. Vol. 96, 1971. In this system, each F1 and back cross-generation will segregate 50/50 for sterility/fertility. The male sterile plants cannot be identified until flowering. In a seed production crop, 50% of the female parent must be rogued. Finally, as noted by Ryder, the dominance of the male-sterile effect would appear to negate this gene's usefulness in a possible F1 hybrid breeding program since it would be extremely difficult to maintain since F1 plants would set no or little seed.

The present methods provide male sterile lines developed from a Salinas, CA-type Iceberg lettuce known to segregate 50/50 for male sterility. The male sterility of the Salinas lettuce has been described as being the result of a single dominant gene.

### Parent of the Male Sterile Line

In October of Year One, two hundred and fifty seeds of line MS7×Salinas 88, BC4 were obtained from Dr. Edward Ryder at the USDA Salinas Calif. research facility. MS7 was first described in Ryder, Genetic Studies in Lettuce (Lactuca sativa L.), J. Amer. Soc. Hort. Sci. (1971) 96(6): 826-828.

In February of Year Two, fifty MS7×Salinas 88, BC4 plants were grown in green-houses to begin further back crosses. Fourteen additional varieties were also planted at this time, including a Salinas 88 selection for use in future crosses. It was noted that the MS7×Salinas 88 BC4 line was not uniform, and that the plants varied for type and maturity. It was further noted that 50% of the plants were early bolting; these were later described as having an “eb” gene. The “eb” gene did not appear to be linked to the sterility gene, as 50% of the early bolters were observed to be sterile, as were 50% of the later maturing plants.

Crosses of Parent with Other Varieties

Sterile late bolting plants were identified and crosses were made with the following lettuce varieties from multiple market classes: Alpine, Napoleon, Sun Devil, Liberty, Icon, Headmaster, Monument, Winterhaven, Avalanche, Supercoach, Delta John, UC2202, UC2205, UC2206. Cuttings were also taken from the late bolting sterile plants and maintained in tissue culture.

### F1 Generation

The F1 seed was harvested in July of Year Two. The F1 seed from each line was then planted in the greenhouse in August of Year Two, and ten MS7×Salinas 88, BC4 F1 lines were planted by hand in a field trial. In all but two of the crosses we noted the presence of the “eb” gene, and thus 50% of the BC4 F1 plants exhibited early bolting behavior. Line 34 (MS7Salinas88BC4×PX839, F1), and line 40 (MS7Salinas88BC4×Salinas88, F1) did not express the early bolting characteristic.

Cuttings from all plants in the non-bolting lines were taken and maintained in tissue culture. At the flowering stage, the sterile plants were identified and noted, particularly 34-1, 34-4, 34-7, 34-12, 34-15, and 40-12. These plants were increased in tissue culture and all other lines were discarded.

### Analysis

The MS7×Salinas 88 BC4 plant which PX 839 and Salinas 88 were crossed onto apparently did not express the “eb” gene as the resultant F1 lines showed no early bolting tendencies. These particular MS7×Salinas 88 BC4 plants used in the above cross and from which cuttings were taken were later described as (MS7×Salinas 88 BC4: MS7 ms7ebeb).

The plants that exhibited the early bolting characteristic were described as (MS7×Salinas88BC4: MS7 ms7Ebeb).

The genotype of the resultant F1 lines from the MS7Salinas88BC4×PX 839 and MS7Salinas 88BC4×Salinas 88 crosses was characterized as: MS7 ms7ebeb

Heterozygous for the dominant sterility gene, and homozygous recessive for the dominant early bolting gene.

More F1 Hybrids by Crossing Non-Bolting Sterile F1 Lines from August of Year Two with Other Lettuce Lines

Cuttings from the individual plants identified as late bolting and male sterile were taken at our research green house facilities and transferred to a laboratory that contracted to perform the tissue culture increases. Once roots were established on the original cuttings, these plants were multiplied and maintained in the tissue culture lab.

The late-bolting, sterile F1 plants selected for tissue culture increases of lines 34-1, 34-4, 34-7, 34-12, 34-15, and 40-12 in August of Year Two were transferred from tissue culture to our greenhouse facility in March of Year Three. Additional lettuce varieties from multiple market classes were also grown from seed at this time to provide pollen for further crossing and back crossing. In June and July of Year Three, crosses of the additional lettuce varieties were made onto all of the male sterile lines, producing a multitude of new F1 hybrids. F1 seed was harvested in July and August of Year Three.

### Back Crosses

A strict back crossing schedule was established to develop new female (male sterile) breeding lines in multiple market classes. In September of Year Three, twenty-five plants from each F1 hybrid were replanted in the greenhouse. The male parent varieties were also planted at this time to provide pollen for back crossing. By February of Year Four, the lines had all begun to flower and the individual F1 male sterile plants from each line were identified. Male fertile F1 plants were removed and destroyed. Back crosses were made on all F1 male sterile plants and BC1 seed was harvested by May of Year Four.

All BC1 seed was cleaned and processed and replanted in the greenhouse facility along with the male fertile parents in June of Year Four. Again the male sterile BC1 plants were identified and their male fertile sibs were removed and destroyed. Additional back crosses were made onto the male sterile BC1 plants. BC2 seed from each line was harvested in November of Year Four.

All BC2 seed was cleaned and processed and replanted in the greenhouse facility along with the male fertile parents in December of Year Four. Additional male fertile lines were also planted to produce BC2 F1 test crosses for evaluation in the summer of Year Five. Again the male sterile BC2 plants were identified and their male fertile sibs were removed and destroyed. Additional back crosses were made onto the male sterile BC2 plants. Multiple test crosses were also made on the BC2 male sterile plants.

BC3 seed and BC2 F1 seed from each line were harvested in May of Year Five. Trials of 40 BC2 F1 individual hybrids were evaluated for yield analysis in summer and fall of Year Five in Salinas, Calif. and again in the winter harvest period in Yuma, Ariz. The BC2 F1 hybrids trialed were both intermarket class hybrids and with-in market class hybrids.

All seed was cleaned and processed and replanted in the greenhouse facility along with the male fertile parents in June of Year Five. Additional male fertile lines were also planted to produce BC3 F1 test crosses for evaluation in the Year Five/Year Six growing seasons. Male sterile BC3 and F1 plants were identified and their male fertile sibs were removed and destroyed.

Additional back crosses were made onto the male sterile BC3 and F1 plants. Multiple test crosses were also made on the BC3 male sterile plants. BC4 seed, BC3F1 seed, and new BC1 seed from each line were harvested in October of Year Five.

The development of new male sterile parent lines has continued to date through multiple generations of back crossing. Uniform BC4, BC5, BC6, BC7 and BC8 male sterile lines have been developed in Iceberg, Romaine, Green Leaf, and Butter market classes.

Comparison of Male Sterile Lines of this Disclosure to Existing Male Sterile Lines

Existing male sterile lines are non-refined, of no commercial value, highly segregating, and not adapted to the commercial lettuce growing regions in California and Arizona, and limited to an Iceberg market class. In contrast, the male sterile lines described herein are of multiple market classes including Iceberg, Romaine, Green Leaf and Butter types and, and are adapted to specific planting locations by virtue of being based on the lines adapted to particular regions and are uniform in type, appearance, adaptability and resistance when applicable, and are commercially viable. Commercial viability is determined by the adaptability and performance of a given lettuce variety. For a lettuce variety to be considered commercially viable the lettuce variety must repeatedly produce yields and a quality product that meets or exceeds the market standards for that planting and harvest period.

### Example 2

### Use of the Male Sterile Lines of this Disclosure for Hybrid Lettuce Seed Production

The use of the male sterile lines of this disclosure in the breeding of new lettuce varieties is described herein. The sterility (lack of pollen) of the female parent eliminates competition between self and foreign pollen, thus increasing the rate of production of intermarket class F1 hybrid seed. The F1 seed is produced as the pollen from the male parent of a specified market class of lettuce is transferred to the pollen free stigmas of the female (male sterile) parent from a different market class.

These lines find particular use in the development of intermarket class hybrid lettuce lines. These lines may be used in any method useful for breeding lettuce varieties. The use of a male sterile line eliminates the need to emasculate one of the parents in order to reduce selfing. For example, instead of washing pollen from stigma and manually transferring pollen to the de-pollinated maternal parent, hybrid seed production can be achieved in a single step by manually transferring pollen to a male sterile parent. In one embodiment, intermarket class hybrid lettuce plants are produced by providing an inbred male sterile line of a specified market class as described herein and another inbred pollen producing line from a distinct market-class.

### Example 3

### Comparative Testing of Lettuce Hybrids Produced by Methods of this Disclosure

Test crosses were made by hand onto BC3, BC4, BC5, BC6 and BC7 male sterile lines from multiple market classes. The intermarket class F1 hybrid test cross seed was harvested, labeled and packaged. Replicated trials were planted multiple times in the Salinas Valley and of California, and in the desert south west production region of Yuma, Ariz.

In many of the trials the test cross F1's and parent varieties were replicated 3 times in each trial, 40 plants in each replication. Each plot was 20 feet in length on raised beds with 40 inch centers, 2 seed lines per bed. The plants were thinned to 12 inches. All treatments were treated identically. Ten continuous plants were cut from each plot (5 from each seed line), and the individual plants were weighed to the nearest gram and their head circumference measured to the nearest 0.5 cm.

The trials were conducted in commercial lettuce fields, and all entries were grown under the same field conditions. None of the material from the trials was commercially harvested, and all remaining plants were disked down following evaluation.

For example, an intermarket class F1 hybrid product is (but is not limited to) the resultant F1 hybrid produced from the hybridization of the following market classes in Table 1:

TABLE 1
Intermarket Class Crosses

| Parent | | | | | | |
|---|---|---|---|---|---|---|
| Types | Iceberg | Romaine | Green Leaf | Red Leaf | Batavia | Butter |
| Iceberg | | I × R | I × GL | I × RL | I × Ba | I × Bu |
| Romaine | R × I | | R × GL | R × RL | R × Ba | R × Bu |
| Green | GL × I | GL × R | | GL × RL | GL × Ba | GL × Bu |
| Leaf | | | | | | |
| Red | RL × I | RL × R | RL × GL | | RL × Ba | RL × Bu |
| Leaf | | | | | | |
| Batavia | Ba × I | Ba × R | Ba × GL | Ba × RL | | Ba × Bu |
| Butter | Bu × I | Bu × R | Bu × GL | Bu × RL | Bu × Ba | |

Each market class lettuce variety has distinctive characteristics based on its genetics. For example, Iceberg lettuce is typically more dense than Romaine lettuce which tends to be more open and leafy. Thus, depending on the characteristics of the two parent varieties being crossed, phenotypic variations occur in the resulting intermarket class F1 hybrids. For example, an Iceberg lettuce crossed with a Romaine lettuce may produce an F1 intermarket class F1 hybrid lettuce plant that is less dense than the Iceberg parent but that is more dense as compared to the Romaine parent.

More subtle phenotypic variations may occur between the F1 hybrids from the same intermarket class cross due to intraspecies variation, such as individual parent plant weight. For instance, there may be variation between the F1 hybrids produced from different Iceberg and Romaine crosses depending on the particular characteristics of the individual parent varieties used. To this end, we have developed, produced and evaluated large numbers of new uniform F1 hybrids from multiple intermarket class crosses. The trial data discussed below illustrates that while there may be variation in phenotypic expression depending on the specific intermarket class, the overall intermarket class crosses of the two market classes consistently produce hybrid plants exhibiting superior size compared to one or both parents.

We have produced seed and grown in field trials multiple Iceberg×Romaine, Iceberg×Green Leaf, Iceberg×Red Leaf, Iceberg×Batavia, Iceberg×Butter, Romaine×Iceberg, Romaine×Green Leaf, Romaine×Red Leaf, Romaine×Batavia, Romaine×Butter, Green Leaf×Iceberg, Green Leaf×Romaine, Green Leaf×Red Leaf, Green Leaf×Batavia, Green Leaf×Butter lettuce hybrids. As mentioned and illustrated below these lettuce hybrids are uniform and distinct.

The data in Tables 2-10 was generated from research trials conducted in Yuma, Ariz., and Salinas, Calif., over a four year period. Each row of the chart represents the average data resulting from a cross between the two listed parents. For each cross, the average head weight is calculated from the average fresh plant weight of 10 plants. Each plant is cut and trimmed to market specifications then weighed to the nearest gram. Heterosis is expressed as the percent fresh head weight increase of the intermarket F1 hybrid vs. each of the parent lines. The range of heterosis from these trials is between 13% and 162% depending on the combining ability of the parent varieties and their performance.

### Iceberg×Romaine

F1 products generated from crossing an Iceberg parent by a Romaine parent yield a range in phenotype depending on the parents selected. The majority of the F1 Iceberg×Romaine products produce some intermediate phenotype, expressing some combined traits of both market classes. In general, an Iceberg×Romaine F1 described herein will be larger and heavier than both the Iceberg and Romaine parent. In all cases, an Iceberg×Romaine F1 described herein will be heavier than at least one of the parents. Also the F1 products are generally looser heading and more elongated than the Iceberg parent, while denser and broader heading than the Romaine parent. The degree of expression is dependent on the combinability of the specific parent varieties crossed. For instance hybrid A002×PM3001 produces a taller, larger plant expressing more characteristics of the romaine parent, where as hybrid A006×PM3012 produced a shorter, denser more spherical plant more closely expressing the traits of the iceberg parent.

In research trials, an Iceberg female crossed with a Romaine male produced an F1 hybrid that, on average, had a 45% increase in fresh head weight at maturity as compared to the female parent, and a 72% increase in fresh head weight at maturity as compared to the male parent (Table 2):

TABLE 2
Iceberg female × Romaine male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| RSV07033 | 642 | 498 | 979 | 52.49 | 96.59 | Iceberg | Romaine |
| RSV07033 | 642 | 498 | 1014 | 57.94 | 103.61 | Iceberg | Romaine |
| RSV07033 | 642 | 425 | 981 | 52.80 | 130.82 | Iceberg | Romaine |
| RSV07033 | 642 | 425 | 1024 | 59.50 | 140.94 | Iceberg | Romaine |
| RSV07033 | 642 | 425 | 1026 | 59.81 | 141.41 | Iceberg | Romaine |
| RSV07033 | 642 | 658 | 918 | 42.99 | 39.51 | Iceberg | Romaine |
| RSV07033 | 642 | 658 | 1001 | 55.92 | 52.13 | Iceberg | Romaine |
| RSV07033 | 642 | 526 | 970 | 51.09 | 84.41 | Iceberg | Romaine |
| RSV07033 | 642 | 526 | 1082 | 68.54 | 105.70 | Iceberg | Romaine |
| RSV05148 | 516 | 536 | 888 | 72.09 | 65.67 | Iceberg | Romaine |
| RSV05148 | 516 | 536 | 955 | 85.08 | 78.17 | Iceberg | Romaine |
| RSV07033 | 758 | 625 | 1007 | 32.85 | 61.12 | Iceberg | Romaine |
| RSV07033 | 758 | 625 | 1091 | 43.93 | 74.56 | Iceberg | Romaine |
| RSV07033 | 758 | 425 | 940 | 24.01 | 121.18 | Iceberg | Romaine |
| RSV07033 | 758 | 425 | 988 | 30.34 | 132.47 | Iceberg | Romaine |
| RSV07033 | 801 | 498 | 954 | 19.10 | 91.57 | Iceberg | Romaine |
| RSV07033 | 801 | 425 | 906 | 13.11 | 113.18 | Iceberg | Romaine |
| RSV07033 | 801 | 425 | 1025 | 27.97 | 141.18 | Iceberg | Romaine |
| RSV05148 | 424 | 536 | 926 | 118.40 | 72.76 | Iceberg | Romaine |
| RSV05148 | 424 | 536 | 1003 | 136.56 | 87.13 | Iceberg | Romaine |
| RSV05148 | 424 | 536 | 1072 | 152.83 | 100.00 | Iceberg | Romaine |
| RSV07033 | 801 | 526 | 946 | 18.10 | 79.85 | Iceberg | Romaine |
| RSV07033 | 801 | 526 | 1010 | 26.09 | 92.02 | Iceberg | Romaine |
| RSV07033 | 856 | 625 | 1077 | 25.82 | 72.32 | Iceberg | Romaine |
| RSV07033 | 856 | 625 | 1172 | 36.92 | 87.52 | Iceberg | Romaine |
| RSV06096 | 835 | 846 | 1459 | 74.73 | 72.46 | Iceberg | Romaine |
| RSV06096 | 835 | 679 | 1205 | 44.31 | 77.47 | Iceberg | Romaine |
| RYM06060 | 747 | 593 | 1122 | 50.20 | 89.21 | Iceberg | Romaine |
| RSV06096 | 835 | 855 | 1210 | 44.91 | 41.52 | Iceberg | Romaine |
| RSV06096 | 835 | 822 | 1147 | 37.37 | 39.54 | Iceberg | Romaine |
| RSV06096 | 835 | 716 | 1266 | 51.62 | 76.82 | Iceberg | Romaine |
| RSV07033 | 856 | 526 | 1075 | 25.58 | 104.37 | Iceberg | Romaine |
| RYM06134 | 432 | 572 | 838 | 93.98 | 46.50 | Iceberg | Romaine |
| RSV05148 | 270 | 536 | 631 | 133.70 | 17.72 | Iceberg | Romaine |
| RSV05148 | 270 | 536 | 692 | 156.30 | 29.10 | Iceberg | Romaine |
| RSV07033 | 876 | 526 | 1080 | 23.29 | 105.32 | Iceberg | Romaine |
| RSV07033 | 876 | 526 | 1112 | 26.94 | 111.41 | Iceberg | Romaine |
| 18-08 | 834 | 628 | 1078 | 29.26 | 71.66 | Iceberg | Romaine |
| 18-08 | 834 | 732 | 957 | 14.75 | 30.74 | Iceberg | Romaine |
| 18-08 | 834 | 653 | 916 | 9.83 | 40.28 | Iceberg | Romaine |
| 18-08 | 834 | 598 | 968 | 16.07 | 61.87 | Iceberg | Romaine |
| 18-08 | 834 | 712 | 1025 | 22.90 | 43.96 | Iceberg | Romaine |
| 18-08 | 834 | 654 | 1002 | 20.14 | 53.21 | Iceberg | Romaine |
| 18-08 | 834 | 714 | 968 | 16.07 | 35.57 | Iceberg | Romaine |
| 18-08 | 834 | 689 | 899 | 7.79 | 30.48 | Iceberg | Romaine |
| 18-08 | 834 | 665 | 1014 | 21.58 | 62.76 | Iceberg | Romaine |
| 18-08 | 834 | 742 | 1102 | 32.13 | 65.71 | Iceberg | Romaine |
| 18-08 | 834 | 623 | 989 | 18.59 | 43.96 | Iceberg | Romaine |
| 18-08 | 834 | 687 | 1025 | 22.90 | 38.14 | Iceberg | Romaine |
| 18-08 | 745 | 628 | 897 | 20.40 | 42.83 | Iceberg | Romaine |
| 18-08 | 745 | 732 | 1035 | 38.93 | 41.39 | Iceberg | Romaine |
| 18-08 | 745 | 653 | 964 | 29.40 | 47.63 | Iceberg | Romaine |
| 18-08 | 745 | 598 | 936 | 25.64 | 56.52 | Iceberg | Romaine |
| 18-08 | 745 | 712 | 925 | 24.16 | 29.92 | Iceberg | Romaine |
| 18-08 | 745 | 654 | 1008 | 35.30 | 54.13 | Iceberg | Romaine |
| 18-08 | 745 | 714 | 993 | 33.29 | 39.08 | Iceberg | Romaine |
| Ave | 724 | 600 | 1009 | 45.83 | 72.63 | | |
| Max | 876 | 855 | 1459 | 156.30 | 141.41 | | |
| Min | 270 | 425 | 631 | 7.79 | 17.72 | | |

A Romaine female crossed with an Iceberg male produced an F1 hybrid that, on average, had a 50% increase in fresh head weight at maturity as compared to the female parent, and a 47% increase in fresh head weight at maturity as compared to the male parent (Table 3):

TABLE 3
Romaine female × Iceberg male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| RSV06096 | 726 | 789 | 1220 | 68.04 | 54.63 | Romaine | Iceberg |
| RYM06146 | 820 | 857 | 975 | 18.90 | 13.77 | Romaine | Iceberg |
| RSV07033 | 658 | 678 | 1045 | 58.81 | 54.13 | Romaine | Iceberg |
| RSV07033 | 658 | 678 | 1101 | 67.33 | 62.39 | Romaine | Iceberg |
| RSV06096 | 726 | 687 | 1309 | 80.30 | 90.54 | Romaine | Iceberg |
| RSV07033 | 658 | 622 | 995 | 51.22 | 59.97 | Romaine | Iceberg |
| RSV07033 | 658 | 622 | 1120 | 70.21 | 80.06 | Romaine | Iceberg |
| RSV06096 | 726 | 879 | 1383 | 90.50 | 57.34 | Romaine | Iceberg |
| RSV06096 | 726 | 852 | 1257 | 73.14 | 47.54 | Romaine | Iceberg |
| RSV07033 | 658 | 489 | 992 | 50.76 | 102.86 | Romaine | Iceberg |
| RSV07033 | 658 | 489 | 1033 | 56.99 | 111.25 | Romaine | Iceberg |
| RYM06134 | 690 | 432 | 883 | 27.97 | 104.40 | Romaine | Iceberg |
| RSV06096 | 726 | 865 | 997 | 37.33 | 15.26 | Romaine | Iceberg |
| RSV07033 | 658 | 738 | 858 | 30.40 | 16.26 | Romaine | Iceberg |
| RSV07033 | 658 | 735 | 934 | 41.95 | 27.07 | Romaine | Iceberg |
| 18-08 | 683 | 855 | 925 | 35.43 | 8.19 | Romaine | Iceberg |
| 18-08 | 683 | 796 | 916 | 34.11 | 15.08 | Romaine | Iceberg |
| 18-08 | 683 | 824 | 859 | 25.77 | 4.25 | Romaine | Iceberg |
| 18-08 | 683 | 754 | 936 | 37.04 | 24.14 | Romaine | Iceberg |
| 18-08 | 683 | 855 | 999 | 46.27 | 16.84 | Romaine | Iceberg |
| 18-08 | 683 | 824 | 1025 | 50.07 | 24.39 | Romaine | Iceberg |
| Ave | 691 | 730 | 1036 | 50 | 47 | | |
| Max | 820 | 879 | 1383 | 90.5 | 111.2 | | |
| Min | 658 | 432 | 858 | 18.9 | 4.2 | | |

### Iceberg×Green Leaf

F1 products from the cross of an Iceberg×Green Leaf produce similar results, again highly dependent on the parent varieties crossed. In general, the F1 products produced will express some intermediate phenotype between the two types of lettuce. In all cases, an Iceberg×Green Leaf F1 described herein will be heavier than at least one of the parents. Iceberg×Green Leaf F1 hybrids are typically non heading with a dense heart. The plants are broad growing and have a more savoyed leaf surface and undulating to frilled leaf margin as compared to the Iceberg parents. The F1 hybrids have denser hearts, larger leaves, and a crispier texture than the Green Leaf parent varieties. The degree of phenotypic expression in the F1 hybrid is dependent on the specific parent varieties used to produce the hybrid.

For the specific crosses in Table 4 below, the female Iceberg parent, on average, was significantly heavier than the male Green Leaf parent. This weight imbalance may have lead to the F1 hybrid being larger and heavier as compared to the Green Leaf male parent variety but not compared to the Iceberg female parent variety. In research trials, an Iceberg female crossed with a Green Leaf male produced an F1 hybrid that, on average, had a 3% decrease in fresh head weight at maturity as compared to the female parent, and a 58% increase in fresh head weight at maturity as compared to the male parent (Table 4):

TABLE 4
Iceberg female × Green Leaf male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| A006a | 806 | 589 | 785 | −2.61 | 33.28 | Iceberg | Green leaf |
| A006a | 806 | 623 | 795 | −1.36 | 27.61 | Iceberg | Green leaf |
| A006a | 806 | 452 | 821 | 1.86 | 81.64 | Iceberg | Green leaf |
| A006a | 806 | 543 | 798 | −0.99 | 46.96 | Iceberg | Green leaf |
| A006a | 806 | 610 | 725 | −10.05 | 18.85 | Iceberg | Green leaf |
| A006a | 806 | 335 | 805 | −0.12 | 140.30 | Iceberg | Green leaf |
| A006a | 806 | 489 | 818 | 1.49 | 67.28 | Iceberg | Green leaf |
| A006a | 806 | 468 | 824 | 2.23 | 76.07 | Iceberg | Green leaf |
| A006a | 806 | 501 | 836 | 3.72 | 66.87 | Iceberg | Green leaf |
| A006a | 806 | 536 | 625 | −22.46 | 16.60 | Iceberg | Green leaf |
| Ave | 806 | 515 | 783 | −3 | 58 | | |
| Max | 806 | 623 | 836 | 3.7 | 140.3 | | |
| Min | 806 | 335 | 625 | −22.5 | 16.6 | | |

A Green Leaf female crossed with an Iceberg male produced an F1 hybrid that, on average, had a 35% increase in fresh head weight at maturity as compared to the female parent, and a 74% increase in fresh head weight at maturity as compared to the male parent (Table 5):

TABLE 5
Green Leaf female × Iceberg male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| RSV07044 | 456 | 410 | 541 | 18.64 | 31.95 | Green Leaf | Iceberg |
| RSV07044 | 456 | 320 | 689 | 51.10 | 115.31 | Green Leaf | Iceberg |
| Ave | 456 | 365 | 615 | 35 | 74 | | |
| Max | 456 | 410 | 689 | 51.1 | 115.3 | | |
| Min | 456 | 320 | 541 | 18.6 | 32.0 | | |

### Iceberg×Red Leaf

F1 products from the cross of an Iceberg×Red Leaf produce similar results to the Iceberg×Green Leaf, and again are highly dependent on the parent varieties crossed. In general, the F1 hybrids will express some intermediate phenotype between the two parental phenotypes of lettuce, and again in a high percentage of cases, the F1 hybrid is larger and heavier than either parent variety. In all cases, an Iceberg×Red Leaf F1 described herein will be heavier than at least one of the parents. Up to six crosses of an Iceberg female with a Red Leaf male were performed. Approximately ten products of each cross were examined. The Iceberg×Red Leaf F1 hybrids are typically non heading with a dense heart, and express some level of red pigmentation. The plants are red and green, broad growing, have a more savoyed leaf surface, and an undulating to frilled leaf margin as compared to the Iceberg parents. The F1 hybrids have denser hearts, larger leaves, less red color, and a crispier texture than the Red Leaf parent varieties. But again the degree phenotypic expression in the F1 hybrid is dependent on the combinability of specific parent varieties used to produce the hybrid.

### Iceberg×Batavia

Iceberg×Batavia F1 hybrids also exhibit some combination of phenotypic traits from both parents. Up to six crosses of an Iceberg female with a Batavia male were performed. Approximately ten products of each cross were examined. The hybrids generated from these combinations generally form heads that are spherical to elongated in shape, and loose to solid in density. The color of the hybrids will vary depending on the parent lines, as will the size and texture. Generally theses hybrid products are larger and heavier than the parent varieties. In all cases, an Iceberg×Batavia F1 described herein will be heavier than at least one of the parents.

### Iceberg×Butter

Iceberg×Butter F1 hybrids also exhibit some combination of phenotypic traits of both parents. The products generated from these combinations generally form spherically shaped heads that range in solidity. The colors will vary depending on the combinability of the parent lines, as will their size and texture. The leaf texture of this particular type of intermarket class hybrid is generally more Buttery than Iceberg, and crisper than a true Butter. Heterosis is also present, and in general the F1 hybrids are typically larger and heavier than the parent varieties. In all cases, an Iceberg×Butter F1 described herein will be heavier than at least one of the parents.

In research trials, an Iceberg female crossed with a Butter male produced an F1 hybrid that, on average, had a 12% increase in fresh head weight at maturity as compared to the female parent and a 61% increase in fresh head weight at maturity as compared to the male parent (Table 6):

TABLE 6
Iceberg female × Butter male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| 17-08 | 785 | 624 | 925 | 17.83 | 48.24 | Iceberg | Butter |
| 17-08 | 785 | 545 | 869 | 10.70 | 59.45 | Iceberg | Butter |
| 17-08 | 785 | 489 | 796 | 1.40 | 62.78 | Iceberg | Butter |
| 17-08 | 785 | 503 | 965 | 22.93 | 91.85 | Iceberg | Butter |
| 17-08 | 785 | 621 | 865 | 10.19 | 39.29 | Iceberg | Butter |
| 17-08 | 785 | 587 | 1006 | 28.15 | 71.38 | Iceberg | Butter |
| 17-08 | 802 | 624 | 958 | 19.45 | 53.53 | Iceberg | Butter |
| 17-08 | 802 | 545 | 947 | 18.08 | 73.76 | Iceberg | Butter |
| 17-08 | 802 | 489 | 875 | 9.10 | 78.94 | Iceberg | Butter |
| 17-08 | 802 | 503 | 957 | 19.33 | 90.26 | Iceberg | Butter |
| 17-08 | 802 | 621 | 936 | 16.71 | 50.72 | Iceberg | Butter |
| 17-08 | 802 | 587 | 887 | 10.60 | 51.11 | Iceberg | Butter |
| 24-07 | 754 | 568 | 802 | 6.37 | 41.20 | Iceberg | Butter |
| 24-07 | 754 | 604 | 898 | 19.10 | 48.68 | Iceberg | Butter |
| 24-07 | 754 | 523 | 916 | 21.49 | 75.14 | Iceberg | Butter |
| 24-07 | 754 | 589 | 796 | 5.57 | 35.14 | Iceberg | Butter |
| 24-07 | 754 | 498 | 884 | 17.24 | 77.51 | Iceberg | Butter |
| 24-07 | 754 | 566 | 826 | 9.55 | 45.94 | Iceberg | Butter |
| 24-07 | 845 | 568 | 935 | 10.65 | 64.61 | Iceberg | Butter |
| 24-07 | 845 | 604 | 869 | 2.84 | 43.87 | Iceberg | Butter |
| 24-07 | 845 | 523 | 997 | 17.99 | 90.63 | Iceberg | Butter |
| 24-07 | 845 | 589 | 925 | 9.47 | 57.05 | Iceberg | Butter |
| 24-07 | 845 | 498 | 936 | 10.77 | 87.95 | Iceberg | Butter |
| 24-07 | 845 | 566 | 845 | 0.00 | 49.29 | Iceberg | Butter |
| 24-07 | 796 | 568 | 879 | 10.43 | 54.75 | Iceberg | Butter |
| 24-07 | 796 | 604 | 932 | 17.09 | 54.30 | Iceberg | Butter |
| 24-07 | 796 | 523 | 916 | 15.08 | 75.14 | Iceberg | Butter |
| 24-07 | 796 | 589 | 798 | 0.25 | 35.48 | Iceberg | Butter |
| 24-07 | 796 | 498 | 912 | 14.57 | 83.13 | Iceberg | Butter |
| 24-07 | 796 | 566 | 916 | 15.08 | 61.84 | Iceberg | Butter |
| Ave | 799 | 560 | 893 | 12 | 61 | | |
| Max | 845 | 621 | 997 | 21.5 | 90.6 | | |
| Min | 754 | 498 | 796 | 0.0 | 35.1 | | |

### Romaine×Green Leaf

F1 hybrids from the cross of a Romaine×Green Leaf produce similar results, again highly dependent on the combinability of the specific parent varieties crossed. In general, the F1 hybrids produced will express some intermediate phenotype between the two phenotypes of the parent varieties, and again, the F1 hybrid is typically larger and heavier than either parent variety. In all cases, a Romaine×Green Leaf F1 described herein will be heavier than at least one of the parents. Romaine×Green Leaf F1 hybrids are typically non heading and have an upright and open growth habit with loose to dense hearts. The plants are broad growing and have a more savoyed leaf surface and undulating to frilled leaf margin as compared to the Romaine parents. The F1 hybrids have larger leaves and a more pronounced mid rib than the Green Leaf parent varieties. But again the degree phenotypic expression in the F1 hybrid is dependent by the specific combinability of the parent varieties used to produce the hybrid.

In research trials, a Romaine female crossed with a Green Leaf male produced an F1 hybrid that, on average, had an 18% increase in fresh head weight at maturity as compared to the female parent, and a 54% increase in fresh head weight at maturity as compared to the male parent (Table 7):

TABLE 7
Romaine female × Green Leaf male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| RSV06096 | 726 | 489 | 1167 | 60.74 | 138.65 | Romaine | Green Leaf |
| RSV06096 | 726 | 425 | 1081 | 48.90 | 154.35 | Romaine | Green Leaf |
| RSV06096 | 726 | 458 | 1202 | 65.56 | 162.45 | Romaine | Green Leaf |
| 19-08 | 568 | 489 | 638 | 12.32 | 30.47 | Romaine | Green leaf |
| 19-08 | 568 | 610 | 714 | 25.70 | 17.05 | Romaine | Green leaf |
| 19-08 | 568 | 335 | 736 | 29.58 | 119.70 | Romaine | Green leaf |
| 19-08 | 568 | 489 | 721 | 26.94 | 47.44 | Romaine | Green leaf |
| 19-08 | 568 | 501 | 662 | 16.55 | 32.14 | Romaine | Green leaf |
| 19-08 | 568 | 536 | 613 | 7.92 | 14.37 | Romaine | Green leaf |
| 19-08 | 602 | 623 | 589 | −2.16 | −5.46 | Romaine | Green leaf |
| 19-08 | 602 | 610 | 624 | 3.65 | 2.30 | Romaine | Green leaf |
| 19-08 | 602 | 335 | 616 | 2.33 | 83.88 | Romaine | Green leaf |
| 19-08 | 602 | 489 | 498 | −17.28 | 1.84 | Romaine | Green leaf |
| 19-08 | 602 | 468 | 569 | −5.48 | 21.58 | Romaine | Green leaf |
| 19-08 | 602 | 501 | 685 | 13.79 | 36.73 | Romaine | Green leaf |
| 19-08 | 602 | 536 | 566 | −5.98 | 5.60 | Romaine | Green leaf |
| Ave | 613 | 493 | 730 | 18 | 54 | | |
| Max | 726 | 623 | 1202 | 65.6 | 162.4 | | |
| Min | 568 | 335 | 498 | −17.3 | −5.5 | | |

A Green Leaf female crossed with a Romaine male produced an F1 hybrid that, on average, had a 25% increase in fresh head weight at maturity as compared to the female parent, and a 3% increase in fresh head weight at maturity as compared to the male parent (Table 8):

TABLE 8
Green Leaf female vs Romaine male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| RSV07044 | 456 | 523 | 711 | 55.92 | 35.95 | Green Leaf | Romaine |
| RSV07044 | 456 | 529 | 688 | 50.88 | 30.06 | Green Leaf | Romaine |
| RSV07044 | 456 | 398 | 687 | 50.66 | 72.61 | Green Leaf | Romaine |
| RSV07044 | 456 | 523 | 719 | 57.68 | 37.48 | Green Leaf | Romaine |
| 19-08 | 524 | 689 | 702 | 33.97 | 1.89 | Green Leaf | Romaine |
| 19-08 | 524 | 702 | 798 | 52.29 | 13.68 | Green Leaf | Romaine |
| 19-08 | 524 | 754 | 653 | 24.62 | −13.40 | Green Leaf | Romaine |
| 19-08 | 524 | 610 | 687 | 31.11 | 12.62 | Green Leaf | Romaine |
| 19-08 | 524 | 663 | 624 | 19.08 | −5.88 | Green Leaf | Romaine |
| 19-08 | 524 | 594 | 669 | 27.67 | 12.63 | Green Leaf | Romaine |
| 19-08 | 524 | 722 | 612 | 16.79 | −15.24 | Green Leaf | Romaine |
| 19-08 | 524 | 728 | 589 | 12.40 | −19.09 | Green Leaf | Romaine |
| 19-08 | 524 | 559 | 697 | 33.02 | 24.69 | Green Leaf | Romaine |
| 19-08 | 524 | 618 | 526 | 0.38 | −14.89 | Green Leaf | Romaine |
| 19-08 | 515 | 714 | 627 | 21.75 | −12.18 | Green Leaf | Romaine |
| 19-08 | 515 | 698 | 701 | 36.12 | 0.43 | Green Leaf | Romaine |
| 19-08 | 515 | 598 | 725 | 40.78 | 21.24 | Green Leaf | Romaine |
| 19-08 | 515 | 476 | 718 | 39.42 | 50.84 | Green Leaf | Romaine |
| 19-08 | 515 | 625 | 615 | 19.42 | −1.60 | Green Leaf | Romaine |
| 19-08 | 515 | 702 | 668 | 29.71 | −4.84 | Green Leaf | Romaine |
| 19-08 | 515 | 715 | 693 | 34.56 | −3.08 | Green Leaf | Romaine |
| 19-08 | 515 | 639 | 734 | 42.52 | 14.87 | Green Leaf | Romaine |
| 19-08 | 515 | 721 | 725 | 40.78 | 0.55 | Green Leaf | Romaine |
| 19-08 | 618 | 689 | 689 | 11.49 | 0.00 | Green Leaf | Romaine |
| 19-08 | 618 | 663 | 612 | −0.97 | −7.69 | Green Leaf | Romaine |
| 19-08 | 618 | 625 | 693 | 12.14 | 10.88 | Green Leaf | Romaine |
| 19-08 | 618 | 639 | 716 | 15.86 | 12.05 | Green Leaf | Romaine |
| 19-08 | 515 | 654 | 678 | 31.65 | 3.67 | Green Leaf | Romaine |
| 19-08 | 515 | 726 | 699 | 35.73 | −3.72 | Green Leaf | Romaine |
| Ave | 537 | 656 | 667 | 25 | 3 | | |
| Max | 618 | 728 | 734 | 42.5 | 50.8 | | |
| Min | 515 | 476 | 526 | −1.0 | −19.1 | | |

### Romaine×Red Leaf

F1 products from the cross of a Romaine×Red Leaf produce similar results to the Romaine×Green Leaf, and again are highly dependent on the parent varieties crossed. In general, the F1 hybrids produced will express some intermediate phenotype between the two market classes of lettuce, and again, heterosis is exhibited, and the F1 hybrid is typically larger and heavier than either parent variety. In all cases, a Romaine×Red Leaf F1 described herein will be heavier than at least one of the parents. Up to six crosses of a Romaine female with a Red Leaf male were performed. Approximately ten products of each cross were examined. Romaine×Red Leaf F1 hybrids are typically non heading and have an upright and open growth habit with loose to dense hearts, and express some level of red pigmentation. The plants are varying degrees of red and green, broad growing, have a more savoyed leaf surface, and an undulating to frilled leaf margin as compared to the Romaine parents. The F1 hybrids are broader, have larger leaves, less red color, and a more pronounced mid rib than the Red Leaf parent varieties. But again the degree phenotypic expression in the F1 hybrid is dependent by the specific parent varieties used to produce the hybrid.

### Romaine×Batavia

Romaine×Batavia F1 hybrids also exhibit some combination of phenotypic traits from both parents. Up to six crosses of a Romaine female with a Batavia male were performed. Approximately ten products of each cross were examined. The products generated from these combinations are generally tall with elongated leaves. The growth habit varies from F1 hybrids as well and they range from open types to types with densely cupping hearty. The colors will vary depending on the parent lines, as will the size and texture. Generally theses hybrid products are larger and heavier than the parent varieties. In all cases, a Romaine×Batavia F1 described herein will be heavier than at least one of the parents.

### Romaine×Butter

Romaine×Butter F1 hybrids also exhibit some combination of phenotypic traits from both parents. The products generated from these combinations generally form elongated hearts that range in solidity, from loosely cupping to densely heading. The colors will vary depending on the parent lines, as will the size and texture. The leaf texture of this type of intermarket class hybrid product is generally more buttery than a Romaine, and crisper than a true Butter. Heterosis is also present, and in general the F1 products are typically larger and heavier than the parent varieties. In all cases, a Romaine×Butter F1 described herein will be heavier than at least one of the parents.

In research trials, a Romaine female crossed with a Butter male produced an F1 hybrid that, on average, had a 36% increase in fresh head weight at maturity as compared to the female parent, and a 37% increase in fresh head weight at maturity as compared to the male parent (Table 9):

TABLE 9
Romaine female × Butter male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| 17-08 | 596 | 624 | 968 | 62.42 | 55.13 | Romaine | Butter |
| 17-08 | 596 | 545 | 653 | 9.56 | 19.82 | Romaine | Butter |
| 17-08 | 596 | 489 | 799 | 34.06 | 63.39 | Romaine | Butter |
| 17-08 | 596 | 503 | 768 | 28.86 | 52.68 | Romaine | Butter |
| 17-08 | 596 | 621 | 862 | 44.63 | 38.81 | Romaine | Butter |
| 17-08 | 596 | 587 | 698 | 17.11 | 18.91 | Romaine | Butter |
| 17-08 | 489 | 624 | 896 | 83.23 | 43.59 | Romaine | Butter |
| 17-08 | 489 | 545 | 678 | 38.65 | 24.40 | Romaine | Butter |
| 17-08 | 489 | 489 | 725 | 48.26 | 48.26 | Romaine | Butter |
| 17-08 | 489 | 503 | 689 | 40.90 | 36.98 | Romaine | Butter |
| 17-08 | 489 | 621 | 623 | 27.40 | 0.32 | Romaine | Butter |
| 17-08 | 489 | 587 | 699 | 42.94 | 19.08 | Romaine | Butter |
| 17-08 | 601 | 624 | 726 | 20.80 | 16.35 | Romaine | Butter |
| 17-08 | 601 | 545 | 853 | 41.93 | 56.51 | Romaine | Butter |
| 17-08 | 601 | 489 | 799 | 32.95 | 63.39 | Romaine | Butter |
| 17-08 | 601 | 503 | 725 | 20.63 | 44.14 | Romaine | Butter |
| 17-08 | 601 | 621 | 679 | 12.98 | 9.34 | Romaine | Butter |
| 17-08 | 601 | 587 | 863 | 43.59 | 47.02 | Romaine | Butter |
| Average | 562 | 562 | 761 | 36 | 37 | | |
| Max | 601 | 624 | 968 | 83.2 | 63.4 | | |
| Min | 489 | 489 | 623 | 9.6 | 0.3 | | |

### Green Leaf×Red Leaf

F1 products from the cross of a Green Leaf×Red Leaf are again highly dependent on the parent varieties crossed. In general, the F1 products produced will express some intermediate phenotype between the two types of lettuce, and again in a high percentage of cases, the F1 hybrid is larger and heavier than either parent variety. In all cases, a Green Leaf×Red Leaf F1 described herein will be heavier than at least one of the parents. Up to six crosses of a Green Leaf female with a Red Leaf male were performed. Approximately ten products of each cross were examined. Green Leaf×Red Leaf F1 hybrids are non heading and have an upright and open growth habit with loose to dense hearts, and express some level of red pigmentation. The plants are a varying degree of red and green in color and since growth habit of both red and Green Leaf are similar, the F1 hybrid products produce are again dependent on the specific combination of parent varieties.

### Green Leaf×Batavia

Green Leaf×Batavia F1 hybrids also exhibit some combination of phenotypic traits from both parents. The products generated from these combinations are generally large, broad and open. Two crosses of a female Green Leaf with a Red Leaf male were performed. Approximately ten products of each cross were examined. The growth habit varies from F1 hybrids as well and they range from open highly frilled types to types that are more densely cupped and smoother texture. The colors will vary depending on the parent lines, as will the size and plant structure. Generally theses hybrid products are larger and heavier than the parent varieties. In all cases, a Green Leaf×Batavia F1 described herein will be heavier than at least one of the parents.

### Green Leaf×Butter

Green Leaf×Butter F1 hybrids also exhibit some combination of phenotypic traits from both parents. The products generated from these combinations generally form elongated hearts that range in solidity, from loosely cupping to densely heading. The colors will vary depending on the parent lines, as will the size, frill of the margin and texture. The leaf texture of this type of intermarket class hybrid product is generally soft and Buttery. Heterosis is also present, and in general the F1 products are typically larger and heavier than the parent varieties. In all cases, a Green Leaf×Butter F1 described herein will be heavier than at least one of the parents.

In research trials, a Green Leaf female crossed with a Butter male produced an F1 hybrid that, on average, had a 61% increase in fresh head weight at maturity as compared to the female parent, and a 20% increase in fresh head weight at maturity as compared to the male parent (Table 10):

TABLE 10
Green Leaf female × Butter male

| | Female | Male | | % Weight | % Weight | | |
|---|---|---|---|---|---|---|---|
| | Parent | Parent | F1 Hybrid | Increase | Increase | Intermarket | |
| | Avg Wt | Avg. Wt | Avg. Wt | Hybrid vs | Hybrid vs | Class | |
| Trial No. | (g) | (g) | (g) | Parent 1 | Parent 2 | Hybrid Type | |
| 17-08 | 410 | 624 | 722 | 76.10 | 15.71 | Green Leaf | Butter |
| 17-08 | 410 | 545 | 724 | 76.59 | 32.84 | Green Leaf | Butter |
| 17-08 | 410 | 489 | 659 | 60.73 | 34.76 | Green Leaf | Butter |
| 17-08 | 410 | 503 | 598 | 45.85 | 18.89 | Green Leaf | Butter |
| 17-08 | 410 | 621 | 658 | 60.49 | 5.96 | Green Leaf | Butter |
| 17-08 | 410 | 587 | 693 | 69.02 | 18.06 | Green Leaf | Butter |
| 17-08 | 425 | 624 | 696 | 63.76 | 11.54 | Green Leaf | Butter |
| 17-08 | 425 | 545 | 725 | 70.59 | 33.03 | Green Leaf | Butter |
| 17-08 | 425 | 489 | 634 | 49.18 | 29.65 | Green Leaf | Butter |
| 17-08 | 425 | 503 | 618 | 45.41 | 22.86 | Green Leaf | Butter |
| 17-08 | 425 | 621 | 644 | 51.53 | 3.70 | Green Leaf | Butter |
| 17-08 | 425 | 587 | 678 | 59.53 | 15.50 | Green Leaf | Butter |
| Ave | 418 | 562 | 671 | 61 | 20 | | |
| Max | 425 | 624 | 725 | 76.6 | 34.8 | | |
| Min | 410 | 489 | 598 | 45.4 | 3.7 | | |

In all cases the reciprocal cross will produce the equivalent result.

1-202. (canceled)

203. An intermarket class hybrid lettuce plant seed derived from a cross between a female lettuce plant of a first market class and a male lettuce plant of a second market class wherein the seed produces an intermarket class hybrid lettuce plant having an average increase in fresh head weight at maturity of at least 15% as compared to the average fresh head weight of the female lettuce plant of the first market class at maturity or as compared to the average fresh head weight of the male lettuce plant of the second market class at maturity.

204. The intermarket class hybrid seed of claim 203 wherein the female lettuce plant is an Iceberg lettuce plant and the male lettuce plant is a Romaine lettuce plant; or wherein the female lettuce plant is an Iceberg lettuce plant and the male lettuce plant is a Green Leaf lettuce plant; or wherein the female lettuce plant is an Iceberg lettuce plant and the male lettuce plant is a Red Leaf lettuce plant; or wherein the female lettuce plant is an Iceberg lettuce plant and the male lettuce plant is a Batavia lettuce plant; or wherein the female lettuce plant is an Iceberg lettuce plant and the male lettuce plant is a Butter lettuce plant; or wherein the female lettuce plant is a Romaine lettuce plant and the male lettuce plant is an Iceberg lettuce plant; or wherein the female lettuce plant is a Romaine lettuce plant and the male lettuce plant is a Green Leaf lettuce plant; or wherein the female lettuce plant is a Romaine lettuce plant and the male lettuce plant is a Red Leaf lettuce plant; or wherein the female lettuce plant is a Romaine lettuce plant and the male lettuce plant is a Batavia lettuce plant; or wherein the female lettuce plant is a Romaine lettuce plant and the male lettuce plant is a Butter lettuce plant; or wherein the female lettuce plant is a Butter lettuce plant and the male lettuce plant is an Iceberg lettuce plant; or wherein the female lettuce plant is a Butter lettuce plant and the male lettuce plant is a Romaine lettuce plant; or wherein the female lettuce plant is a Butter lettuce plant and the male lettuce plant is a Green Leaf lettuce plant; or wherein the female lettuce plant is a Butter lettuce plant and the male lettuce plant is a Red Leaf lettuce plant; or wherein the female lettuce plant is a Butter lettuce plant and the male lettuce plant is a Batavia lettuce plant; or wherein the female lettuce plant is a Red Leaf lettuce plant and the male lettuce plant is an Iceberg lettuce plant; or wherein the female lettuce plant is a Red Leaf lettuce plant and the male lettuce plant is a Romaine lettuce plant; or wherein the female lettuce plant is a Red Leaf lettuce plant and the male lettuce plant is a Green Leaf lettuce plant; or wherein the female lettuce plant is a Red Leaf lettuce plant and the male lettuce plant is a Batavia lettuce plant; or wherein the female lettuce plant is a Red Leaf lettuce plant and the male lettuce plant is a Butter lettuce plant; or wherein the female lettuce plant is a Green Leaf lettuce plant and the male lettuce plant is an Iceberg lettuce plant; or wherein the female lettuce plant is a Green Leaf lettuce plant and the male lettuce plant is a Romaine lettuce plant; or wherein the female lettuce plant is a Green Leaf lettuce plant and the male lettuce plant is a Red Leaf lettuce plant; or wherein the female lettuce plant is a Green Leaf lettuce plant and the male lettuce plant is a Batavia lettuce plant; or wherein the female lettuce plant is a Green Leaf lettuce plant and the male lettuce plant is a Butter lettuce plant; or wherein the female lettuce plant is a Batavia lettuce plant and the male lettuce plant is an Iceberg lettuce plant; or wherein the female lettuce plant is a Batavia lettuce plant and the male lettuce plant is a Romaine lettuce plant; or wherein the female lettuce plant is a Batavia lettuce plant and the male lettuce plant is a Green Leaf lettuce plant; or wherein the female lettuce plant is a Batavia lettuce plant and the male lettuce plant is a Red Leaf lettuce plant; or wherein the female lettuce plant is a Batavia lettuce plant and the male lettuce plant is a Butter lettuce plant.

205. The intermarket class hybrid lettuce plant seed of claim 203, wherein the seed produces an intermarket class hybrid lettuce plant having an average increase in fresh head weight at maturity of at least 20%, at least 25%, at least 30%, at least 35%, at least 40%, at least 45%, at least 50%, at least 55%, at least 60%, at least 65%, or at least 70% as compared to the average fresh head weight of the female lettuce plant of the female market class at maturity or the average fresh head weight of the male lettuce plant of the second market class at maturity.

206. An intermarket class hybrid lettuce plant or part thereof produced by growing the seed of claim 203.

207. A lettuce plant having all of the physiological and morphological characteristics of the intermarket class hybrid lettuce plant of claim 206.

208. Pollen of the intermarket class hybrid lettuce plant of claim 206.

209. A tissue culture of the intermarket class hybrid lettuce plant of claim 206.

210. A method for producing intermarket class hybrid lettuce seed, comprising the steps of:
a) providing a first inbred lettuce plant selected from the group of lettuce market classes consisting of Iceberg, Romaine, Green Leaf, Red Leaf, Batavia and Butter lettuce plants wherein the first inbred lettuce plant comprises pollen;
b) providing a second inbred lettuce plant selected from the group of lettuce market classes consisting of Iceberg, Romaine, Green Leaf, Red Leaf, Batavia and Butter lettuce plants wherein the second inbred lettuce plant is emasculated;
c) releasing Megachile bees, wherein the bees transfer pollen from the first inbred lettuce plant to the second inbred lettuce to produce a pollinated inbred lettuce plant wherein the first inbred lettuce plant is from a different market class than the market class of the second inbred lettuce plant; and
d) growing the pollinated lettuce plant to produce hybrid lettuce seed, wherein the intermarket class hybrid lettuce seed produces an intermarket class hybrid lettuce plant having an average increase in fresh head weight at maturity of at least 20%, at least 25%, at least 30% or at least 35% compared to the average fresh head weight at maturity of the first inbred lettuce plant.

211. The method of claim 210, wherein the Megachile bee is selected from the group consisting of: Megachile rotundata, Megachile relativa, and Megachile pugnata.

212. The method of claim 211, wherein the Megachile bee is Megachile rotundata.

213. The method of claim 212, wherein the first lettuce plant is an Iceberg lettuce plant and the second lettuce plant is a Romaine lettuce plant; or wherein the first lettuce plant is an Iceberg lettuce plant and the second lettuce plant is a Green Leaf lettuce plant; or wherein the first lettuce plant is an Iceberg lettuce plant and the second lettuce plant is a Red Leaf lettuce plant; or wherein the first lettuce plant is an Iceberg lettuce plant and the second lettuce plant is a Batavia lettuce plant; or wherein the first lettuce plant is an Iceberg lettuce plant and the second lettuce plant is a Butter lettuce plant; or wherein the first lettuce plant is a Romaine lettuce plant and the second lettuce plant is an Iceberg lettuce plant; or wherein the first lettuce plant is a Romaine lettuce plant and the second lettuce plant is a Green Leaf lettuce plant; or wherein the first lettuce plant is a Romaine lettuce plant and the second lettuce plant is a Red Leaf lettuce plant; or wherein the first lettuce plant is a Romaine lettuce plant and the second lettuce plant is a Batavia lettuce plant; or wherein the first lettuce plant is a Romaine lettuce plant and the second lettuce plant is a Butter lettuce plant; or wherein the first lettuce plant is a Butter lettuce plant and the second lettuce plant is an Iceberg lettuce plant; or wherein the first lettuce plant is a Butter lettuce plant and the second lettuce plant is a Romaine lettuce plant; or wherein the first lettuce plant is a Butter lettuce plant and the second lettuce plant is a Green Leaf lettuce plant; or wherein the first lettuce plant is a Butter lettuce plant and the second lettuce plant is a Red Leaf lettuce plant; or wherein the first lettuce plant is a Butter lettuce plant and the second lettuce plant is a Batavia lettuce plant; or wherein the first lettuce plant is a Red Leaf lettuce plant and the second lettuce plant is an Iceberg lettuce plant; or wherein the first lettuce plant is a Red Leaf lettuce plant and the second lettuce plant is a Romaine lettuce plant; or wherein the first lettuce plant is a Red Leaf lettuce plant and the second lettuce plant is a Green Leaf lettuce plant; or wherein the first lettuce plant is a Red Leaf lettuce plant and the second lettuce plant is a Batavia lettuce plant; or wherein the first lettuce plant is a Red Leaf lettuce plant and the second lettuce plant is a Butter lettuce plant; or wherein the first lettuce plant is a Green Leaf lettuce plant and the second lettuce plant is an Iceberg lettuce plant; or wherein the first lettuce plant is a Green Leaf lettuce plant and the second lettuce plant is a Romaine lettuce plant; or wherein the first lettuce plant is a Green Leaf lettuce plant and the second lettuce plant is a Red Leaf lettuce plant; or wherein the first lettuce plant is a Green Leaf lettuce plant and the second lettuce plant is a Batavia lettuce plant; or wherein the first lettuce plant is a Green Leaf lettuce plant and the second lettuce plant is a Butter lettuce plant; or wherein the first lettuce plant is a Batavia lettuce plant and the second lettuce plant is an Iceberg lettuce plant; or wherein the first lettuce plant is a Batavia lettuce plant and the second lettuce plant is a Romaine lettuce plant; or wherein the first lettuce plant is a Batavia lettuce plant and the second lettuce plant is a Green Leaf lettuce plant; or wherein the first lettuce plant is a Batavia lettuce plant and the second lettuce plant is a Red Leaf lettuce plant; or wherein the first lettuce plant is a Batavia lettuce plant and the second lettuce plant is a Butter lettuce plant.
